(19) United States
(12) Patent Application Publication
(10) Pub. No.: US 2009/0312696 A1
(43) Pub. Date: Dec. 17, 2009

(54) Devices, Systems, and Related Methods for Delivery of Fluid to Tissue

(76) Inventors: Vincent G. Copa, Minnetonka, MN (US); Suranjan Roychowdhury, Minnetonka, MN (US); Sidney F. Hauschild, Minnetonka, MN (US); Justin M. Crank, Minnetonka, MN (US)

Correspondence Address: AMS RESEARCH CORPORATION, 10700 BREN ROAD WEST, MINNETONKA, MN, 55343, US

(21) Appl. No.: 12/087,231

(22) PCT Filed: Dec. 28, 2006
(86) PCT No.: PCT/US2006/049441

§ 371 Date: Nov. 24, 2008

Related U.S. Application Data
(60) Provisional application No. 60/754,730, filed on Dec. 28, 2005.
(60) Provisional application No. 60/856,035, filed on Nov. 2, 2006.
(60) Provisional application No. 60/866,741, filed on Nov. 21, 2006.

(51) Int. Cl. A61M 37/00; A61M 25/10; A61M 3/02

(52) U.S. Cl. 604/43; 604/131; 604/96.01; 604/517

(57) ABSTRACT

Described are devices useful to deliver fluid to tissue of the bladder, bladder neck, prostate, urethra, ureter, kidney, and related methods, the devices including one or a combination of features such as a steerable shaft, an optical mechanism, multiple fluid delivery orifices that may be moveable or extendable laterally, longitudinally, or distally; the methods allowing for delivery of one or multiple types of fluid to tissue such as the bladder (including the bladder neck), urethra, prostate, kidney, ureter, etc.

### PRIORITY CLAIM

The present non-provisional patent Application claims priority under 35 USC §119(e) from U.S. Provisional Patent Application having Ser. No. 60/754,730, filed on Dec. 28, 2005, by Copa et al. and titled DEVICES, SYSTEMS, AND RELATED METHODS FOR DELIVERY OF FLUID TO TISSUE; U.S. Ser. No. 60/856,035, filed Nov. 9, 2006, by Crank et al., entitled MECHANICAL VOLUME CONTROL FOR INJECTION DEVICE, and to U.S. Ser. No. 60/866,741, filed Nov. 21, 2006, by Crank, entitled INJECTION TUBE FOR JET INJECTION DEVICE; wherein the entireties of said provisional patent applications are incorporated herein by reference.

### FIELD OF THE INVENTION

The invention relates to methods and devices for treating tissue of the urinary tract (e.g., prostate tissue, kidneys, ureters, urethral tissue, bladder, etc.), as well as devices, methods, and surgical kits for use in a treatment regimen.

### BACKGROUND

Urinary tract health is an increasingly important health issue, e.g., based on an aging population. Treatment of urinary tract conditions is an area of much investigation.

Many methods and devices have been proposed to deliver therapeutic materials such as therapeutic fluid to the urinary tract, e.g., kidneys, ureters, or lower urinary tract (urethra, prostate, bladder, bladder neck), examples of these devices focusing on treatment of the prostate. Prostate disease is a significant health risk for males. Diseases of the prostate include prostatitis, benign prostatic hyperplasia (BPH, also known as benign prostatic hypertrophy), prostatic intraepithelial neoplasia (PIN), and prostatic carcinoma.

In addition to prostate conditions, other tissue of the urinary tract can be affected by medical conditions that can be treated by delivery of various therapeutic materials in the form of fluids. Tissues of the bladder (which includes the bladder neck), ureter, kidneys, urethra, as well as the prostate, can be treated by delivery of drugs or other therapeutic agents:

Various treatments of the bladder that are currently used or proposed, such as transurethral administration of an active pharmaceutical agent, involve placement of a therapeutic fluid into the bladder using a single needle located at the distal end of a rigid shaft inserted into the bladder through the urethra. The use of a single needle at the distal end of a rigid shaft to inject a therapeutic fluid such as a drug, into the bladder, can involve various difficulties or undesired effects and can be a difficult procedure as well. A rigid shaft with a single needle used to inject tissue of the bladder must be maneuvered, twisted, turned, etc., into position to place the needle at a desired position or multiple positions for multiple fluid deliveries and to apply pressure to the distal end location of the needle.

Therapeutic agents should be delivered with minimized discomfort and procedure time, and with the best degree of accuracy of delivery location and delivery volume as possible. As such, there exists continuing need to provide improved devices for delivering therapeutic fluids to the lower urinary tract, kidneys, ureters, etc.

### SUMMARY

The invention involves multi-functional fluid (e.g., drug or other therapeutic agent) delivery devices. These devices allow for localized delivery of biologically active species and agents, including chemical and biochemical agents, at locations in the male or female urinary tract, e.g., bladder, bladder neck, kidney, ureters, urethra, prostate, etc. The device allows delivery of agents at various tissue locations, also multiple different tissue locations, within the lower urinary tract, kidney, and ureter, using a single device. Devices and methods are useful to provide infusions, injections, or instillations of pharmacological, chemical, and biologic agents for treatment of various urological disease states. The devices can be capable of delivery of precise amounts of fluid for injection or instillation, at precise locations, for improved treatment based on precision and accuracy of fluid delivery.

The multi-purpose versatile transurethal drug delivery devices allow agents that can impact biologic activity, such as pharmaceuticals, proteins, genes, chemicals, and cells, to be accurately delivered to one or more localized areas of the lower urinary tract such as to the bladder (“bladder” as used herein includes the bladder neck), or into the ureters, kidney, prostate, urethra, etc.

The device can provide for multiple and various controllable depths of tissue penetration, multiple arcs, and multiple “throws” (i.e., depths of penetration and localization of fluid volume into the target tissue) for delivery of the same or different types of fluid. The device can include design features that allow for improved placement and accuracy of fluid delivery in terms of location and volume of fluid delivery, and improved patient comfort and safety, such as one or more of a flexible or rigid shaft; optional fluid drainage capabilities for draining urine; the ability to steer a distal end of the device shaft to provide improved precision of location of delivery; an optical feature that allows the user to access a view taken at the distal end of the device, which includes one or more of a view of tissue and an extended delivery orifice; multiple fluid delivery orifices; extendable fluid delivery orifices; multiple fluid delivery systems to allow delivery of two or more different fluids; or any combination of these features depending on the requirements of patient comfort and treatment efficacy.

Advantages of a device can include ease and accuracy of delivery of agents for the physician, with the potential of being a means for in-office treatment of various male and female urological disease states ranging from strictures, urinary tract infections, BPH, prostatitis, overactive bladder, and ureteric inflammation and blockages. Also, the devices can result in better patient comfort and recovery.

General features of certain embodiments of devices can include one or more of the following: improved comfort and utility compared to usefulness of flexible and rigid cystoscopes or delivery devices; high delivery efficiency; in-office treatment capabilities; multiple deliveries, injections, arcs, and “throws” (see definition of “throw” supra) with high reservoir capability; high versatility—functionality across entire urinary tract and bladder and capability of delivery of multiple different agents or multiple volumes of the same or different agents, e.g., at different locations.

According to certain embodiments, the device can be a substantially self-contained device comprising a shaft having a proximal and a distal end, with fluid delivery orifices at the distal end and a body at the proximal end. The device, including the body, may include features such as optics connected to the distal end; a fluid reservoir in communication with the fluid delivery orifices; a pressure source in communication with the fluid reservoir; a light source for illuminated use of the optics; and related mechanisms such as actuators, triggers, etc. for actuating a distal feature such as a proximal steering actuator to cause the distal end to be steered, a proximal trigger to move an orifice extension, or a proximal trigger to cause delivery of fluid. Alternate embodiments can place a fluid reservoir and pressure source at the distal end of the device, proximal to the fluid delivery orifice. Still other alternate embodiments can place the pressure source, fluid reservoir, or both, remote from the proximal end of the device, such as at a remote console. The remote console can connect to a device proximal end or a body by a port at the proximal end or body, the port being in fluid communication with one or more fluid delivery orifices.

Exemplary devices according to the invention, for delivery of fluid to tissue of the lower urinary tract, kidney, ureter, etc., can include a proximal end, a flexible shaft extending from the proximal end to a distal end of the shaft, and a fluid delivery orifice at the distal end of the shaft, the fluid delivery orifice being capable of being extended from the shaft. The fluid delivery orifice may be a needle or a needleless fluid delivery orifice.

The device may include delivery orifices that are one or multiple needles or needleless fluid delivery orifices each of which may be independently extended and retracted from the shaft.

A device, in combination with any other feature described herein, may include multiple needleless delivery orifices located along a length of the shaft, each delivery orifice being independently capable of ejecting a fluid for injection to tissue or instillation at a tissue surface.

A device, in combination with any other feature described herein, may include multiple deliver orifices located at positions around a perimeter of the shaft, each fluid delivery orifice being independently capable of ejecting a fluid for injection to tissue or instillation at a tissue surface.

A device, in combination with any other feature described herein, may include a drainage lumen extending from a distal end to a proximal end, the drainage lumen being capable of draining urine from the bladder when the device is installed in a patient.

A device, in combination with any other feature described herein, may include a balloon or other locating mechanism at the distal end for location within a bladder or bladder neck during use.

A device, in combination with any other feature described herein, may include one or multiple fluid reservoirs and pressure sources located at a distal end, a proximal end, along a length 6f the shaft, or any combination thereof Each of multiple fluid reservoirs may be associated with a delivery orifice to allow delivery of different fluids.

A device may contain one or more needleless fluid delivery orifices that extend from the shaft device on an orifice extension located along the shaft or at a distal end (tip) of the device.

An exemplary device may include a combination of features discussed above such as a body at a proximal end, a flexible (e.g., steerable) shaft extending from the body to a distal end of the shaft, multiple fluid delivery orifices at the distal end of the shaft in fluid communication with one or more fluid reservoirs, one or more multiple pressure sources in communication with one or multiple fluid reservoirs, with fluid delivery orifices located at extendible members that can be extended and retracted from the shaft, along the length of the device, or beyond the distal end (tip) of the device.

As used herein, the term “transurethral,” as in a transurethral fluid delivery method, means a procedure that is performed through or by way of the urethra by administering a fluid delivery device through the inner space of the urethral lumen; the device can enter the urethral lumen through the meatus (male or female) or through the perineum, and a distal end of the device passes through a length of the urethral lumen to deliver a fluid at a location of the lower urinary tract, kidney, ureter, etc.

In one aspect the invention relates to a device for delivery of fluid to tissue of the urinary tract. The device includes a proximal end, a flexible shaft extending from the proximal end to a distal end of the shaft, and an extendable fluid delivery orifice at the distal end, the fluid delivery orifice being capable of being extended from the shaft.

In another aspect the invention relates to methods of delivering fluid to tissue of the urinary tract. Methods include providing a device as described herein, inserting the distal end into the urethra to place a fluid delivery orifice at a location of the urinary tract, extending an extendable fluid delivery orifice, and delivering fluid through the extended orifice to the urinary tact.

In another aspect, the invention relates to a device for delivery of fluid to tissue of the urinary tract. The device includes a proximal end, a shaft extending from the proximal end to a distal end, the shaft comprising a steerable portion, a fluid delivery orifice at the distal end, and optics to allow optical communication between the proximal end and the distal end. Another aspect relates to a method of delivering fluid to tissue of the urinary tract by use of this embodiment of a device as described herein immediately above. The method includes inserting the distal end of the device into the urethra to place a fluid delivery orifice at a location of the urinary tract, viewing a delivery location by use of the optics, steering the distal end, and delivering fluid to the urinary tact.

Another aspect of the invention relates to a device for delivery of fluid to tissue of the urinary tract wherein the device is capable of delivering two or more different fluids. The device includes a proximal end; a shaft extending from the proximal end to a distal end of the shaft; a first set of two or more fluid delivery orifices at the distal end, the fluid delivery orifices in fluid communication with each other and in fluid communication with a first fluid reservoir, a second set of two or more second fluid delivery orifices at the distal end, the second fluid delivery orifices in fluid communication with each other and in fluid communication with a second fluid reservoir; a first cover to selectively open and close one or more first fluid delivery orifice; a second cover to selectively open and close one or more second fluid delivery orifice, and a pressure source capable of pressurizing a fluid reservoir, e.g., to independently delivery fluid from fluid delivery orifices. Related methods involve providing a device as described herein and immediately above and inserting the distal end into the urethra to place a fluid delivery orifice at a location of the urinary tract, and delivering multiple fluids from the distal end.

### BRIEF DESCRIPTION OF THE DRAWINGS

FIG. 1 schematically illustrates a device of the invention, including extendable fluid delivery orifices.

FIG. 1A schematically illustrates a moveable component of device of the invention.

FIG. 2 schematically illustrates a device of the invention, including extendable fluid delivery orifices.

FIG. 3 schematically illustrates a device of the invention, including extendable fluid delivery orifices.

FIG. 4 schematically illustrates a device of the invention, including multiple, independently functioning, fluid delivery orifices.

FIG. 5 schematically illustrates a device of the invention, including multiple, independently functioning, fluid delivery orifices.

FIGS. 6A and 6B schematically illustrate a device of the invention including multiple fluid delivery orifices.

FIGS. 7A and 7B schematically illustrate a device of the invention including multiple fluid delivery orifices.

FIGS. 8A and 8B schematically illustrate a device of the invention, including multiple extendable sets of fluid delivery orifices.

FIGS. 9A, 9B, and 9C schematically illustrate devices of the invention, including multiple extendable fluid delivery orifices or sets of fluid delivery orifices.

FIGS. 10A and 10B schematically illustrate devices of the invention, including multiple extendable fluid delivery orifices.

FIG. 11 schematically illustrates a device of the invention, including multiple extendable fluid delivery orifices.

FIG. 12 schematically illustrates a device of the invention, including a steerable distal end portion.

FIG. 13 schematically illustrates a device of the invention, including a steerable distal end portion and a remote console.

FIG. 14 is a cut-away, side view of an embodiment of a delivery volume control.

FIG. 15 is an end view of the delivery volume control of FIG. 14 taken at line 15-15 of FIG. 14.

### DETAILED DESCRIPTION

The invention relates to devices useful for delivering (e.g., injecting or instilling) fluid to tissue at or near the lower urinary tract, e.g., tissue of the prostate, kidneys, ureters, urethral tissue, bladder (including the bladder neck), etc. The devices eject a therapeutic “fluid” from a distal end of an elongate shaft inserted into the urethra The devices can include multiple orifices that may be stationary or moveable relative to a shaft of the device, for ejecting a fluid at multiple locations. Embodiments of designs that include multiple orifices can include an extended, expanded, or extendable chain, string, array, or sequence (e.g., “daisy chain”). Orifices may be located at an extension mechanism (“orifice extension”) such as extendable or fanning needles or needleless fluid delivery orifices, a balloon that contains needles or needleless injection or ejection mechanisms for delivery of fluid around an inside of a bladder, and the like.

The invention relates to devices, systems, and methods for delivery (e.g., ejection, injection, or instillation) of a fluid into, onto, or otherwise into contact with tissue at or near the lower urinary tract such as the bladder. The systems can overcome undesired or disadvantageous features of systems and methods that use a single needle at a distal end of a rigid shaft, e.g., as have been used for transurethral fluid deliveries of fluid into tissue of the bladder or bladder neck.

The injections can be carried out by a needleless fluid delivery system (e.g., needleless injector system) or using needles. A needleless injector system can include a source of fluid that can be pressurized to cause an injectate (fluid) to penetrate into tissue. Alternately, the fluid may flow from the needle or needleless fluid delivery system at a relatively low pressure to just flow out of the orifice and contact a surface of tissue without significantly penetrating tissue, i.e., by “instillation” of fluid at a surface of a tissue.

Embodiments of devices include multiple fluid delivery orifices, such as in the form of needleless or needle-type fluid delivery orifices arranged according to any useful configuration. Exemplary multiple fluid delivery orifices may be arranged along a length of a device shaft, all at a distal end; may extend or splay out from the shaft at a desired location along the length of the shaft or from the distal end; and may be designed to make sequential or simultaneous multiple fluid deliveries (of the same or different fluids) at specific locations of the anatomy of the urinary tract, e.g., at multiple locations around the urethra, bladder neck, or bladder tissue.

According to exemplary embodiments, a fluid delivery orifice can move in one or multiple dimensions with reference to a longitudinal axis of a shaft A fluid delivery orifice can be capable of moving longitudinally in a direction along a longitudinal axis of the shaft, e.g., along a length of the shaft or “distally” from the end or “tip” of the shaft. In this exemplary embodiment a fluid delivery orifice can extend from a distal end (or “tip”) of the shaft in the direction of a longitudinal axis that includes the distal end. Alternately or in combination, a fluid delivery orifice can be capable of moving laterally away from the shaft, at a location along a length of the shaft or distal from the distal end (“tip”) of the shaft. The fluid delivery orifice may be a component of a moveable orifice extension (e.g., mechanical paddle, needle, lumen, balloon, membrane, or the like) that can be extended and retracted from a position along side of or within the shaft at a position along the length of the shaft, or that can be extended from the distal end (“tip”) of the shaft.

Features of inventive fluid delivery devices are included as part of the present description and may be included in a fluid delivery device individually or in any desired combination. For example, embodiments of the invention may include fluid delivery devices that include positioning features (e.g., “locating mechanisms”) that facilitate proper positioning of a fluid delivery device, and therefore positioning of a fluid delivery orifice (needle or needleless injector) near desired tissue for fluid delivery. Positioning features are various in nature and may include one or more of: a balloon or multiple balloons located at the distal end of the device for placement and fixing the distal end of the device; multiple orifices; moveable orifices; demarcations at a proximal end of a device of distances to distal end features; and an optical feature such as those used to position an endoscope, e.g., or optical fiber. See, e.g., Assignee's copending U.S. patent application Ser. No. 11/186,218, entitled “NEEDLELESS DELIVERY SYSTEMS,” filed Jul. 21, 2005, published as US Patent Publication Number 2006/0129125, the entire disclosure of which is incorporated herein by reference.

Other embodiments of fluid delivery devices may include any one or more of the above features along with one or more tissue tensioners that contact and optionally place pressure on tissue at a desired location relative to a fluid delivery orifice, and optionally can also place a strain or tension on the tissue as desired for delivery of an injection at the surface of the tissue. See, e.g., Assignee's copending United States Patent Publication Number 2006-0129125, the entirety of which is incorporated herein by reference. Examples of tissue tensioners include inflatable or extendable features such as balloons or mechanically extendable features such as paddles, metal cages, other mechanically extendable structure or protrusions, vacuum, etc.

Fluid delivery devices as described can be used with various delivery methods such as methods that allow for direct vision of a fluid injection or instillation wherein an internal location of a fluid delivery orifice is determined visually, and methods referred to as blind delivery methods wherein location of a fluid delivery orifice is determined indirectly.

Direct vision methods involve the use of an optical feature to view a delivery site directly, such as by use of an optical mechanism of the type used with endoscope devices, e.g., optical fiber, included in a fluid delivery device, e.g., as a component of the shaft. In general, an optical feature or optical mechanism may be any optical structure that can be placed in a shaft (e.g., a flexible shaft) to allow viewing at a location of distal end from the proximal end. Useful flexible fiber optic cables are known and commercially available and may be made, e.g., from glass or light carrying flexible polymeric materials. Optionally and preferably a light source can be located at or in optical communication with a distal end.

In one embodiment, a light bulb or other light source (light emitting diode) may be at the distal end, or at a proximal end and connected to the distal end by fiber optic cable; a second optical fiber between the distal end and the proximal end carries light back for viewing at the proximal end. The viewing optical cable may be connected to a lens or an electronic image-capturing device such as a camera or computer. In an exemplary embodiment an electronic image sensor in the form of a miniaturized camera or electronic camera chip (e.g., a charge-coupled device or “CCD” chip) and light source may be placed at the distal end and can be connected electronically to the proximal end to deliver images from the distal end electronically.

A device that allows for blind delivery can instead include one or more non-optical features that allow an operator (e.g., surgeon) to identify the position of a device, and in particular a fluid delivery orifice, e.g., within the urethra, bladder, or bladder neck, etc., so that a fluid delivery for injection or instillation can be performed at a desired location. Blind delivery techniques can identify a delivery location based on features of the device such as a length-measuring feature such as demarcations at the proximal end of the device that reference distances to locations of features at the distal end, by using demarcations in combination with known dimensions of a device and of relevant anatomy. Demarcations may be used also in combination with measurement of anatomical features such as the length of the prostate, urethra, bladder, bladder neck, etc., e.g., by known techniques including those that use ultrasound position measuring equipment. Blind delivery techniques can also involve other features of devices as described herein such as positioning features (e.g., “a locating mechanism” such as a paddle, extension, or balloon at the distal end of the device) and moveable fluid delivery orifices.

Various embodiments of fluid delivery devices of the invention can include different types of shafts, including a flexible shaft, a steerable shaft (considered a “flexible shaft,” a rigid shaft, a multi-piece shaft designed to be assembled and disassembled prior to or following use, an integral shaft that is not designed to be assembled and disassembled prior to or after use, and combinations of these. Particular devices and methods of the invention involve shafts that are flexible integral shafts wherein the device does not include an optical device such as an endoscope but includes positioning features such as a balloon, and is used with blind delivery methods. Other devices and methods involve multi-component shafts that include an endoscope and other features as described herein. Still other embodiments involve an integral shaft that is steerable.

A “steerable” shaft refers to a shaft that is semi-rigid, but still “flexible,” that can be controlled (i.e., steered, articulated, deflected, or controllably bent) in two or three dimensions at its distal end by manipulation of one or more steering actuators at a proximal end of the device. A steering mechanism may involve different mechanical designs, with an example allowing movement in two dimensions based on differential pushing and pulling (or tension and compression) of multiple cables within walls of a shaft. More than one of these mechanisms may be included in a single shaft to allow for movement of an end of a shaft in three dimensions.

Steerable shafts and mechanisms are known and understood in the art of endoscope and other medical devices, and may be designed to allow movement in two or three dimensions to deflect a length of the far distal end or “tip” of the shaft, a desired amount, e.g., at least 45 degrees from straight back toward a proximal end of the device, such as at least 90 degrees, or at least 180 degrees. The radius of curvature of the bend produced upon deflecting the tip can be as desired and may be dependent on the overall design of the shaft and steering mechanism, and desired application of the device. According to one embodiment, the radius of curvature can be one that allows a portion of distal shaft having a length of from 0.5 to 2 inches (e.g., 0.6 to 1 inch) to bend back 180 degrees within the bladder

Generally, the length of the steerable portion can be as desired, e.g., the end 2 inches of a shaft, such as from 0.5 to 1.8 inches, or from 0.6 to 1 inch. Typically, a steerable portion of a shaft can articulate in two dimensions, such as along hinged connections. According to certain embodiments, a shaft may have two or more steerable portions such as two portions that are steerable each in two dimensions, and those dimensions being in orthogonal planes. For example a the end 0.5 to 2 inches (e.g., 0.6 to 1 inch) of a shaft may be steerable in a first two dimensions, and the adjacent 0.5 to 2 inches (e.g., 0.6 to 1 inch) of the shaft may be steerable in a second two dimensions, optionally the second two dimensions can define a plane that is orthogonal to a plane defined by the first two dimensions.

Examples of steering mechanisms include those that cause steering (i.e., articulation, deflection, etc.) of a tip or portion of a shaft by manipulation of multiple hinged deflection points along a length of a shaft, the hinges being manipulated by multiple control wires to bend or deflect the shaft in two dimensions. See, e.g., United States patent publication 2006/00784383, Ser. No. 10/955,930; United States patent publication 2006/0241564, Ser. No. 11/398,890; U.S. Pat. No. 6,610,007, Ser. No. 09/969,927; the entireties of each of these being incorporated herein by reference. As noted, one or more than one of these mechanisms may be included along one or more different portions of length of a steerable shaft to allow steering of one or multiple shaft portions in two or multiple dimensions.

Various embodiments of the invention can optionally or alternately include safety features that prevent inadvertent or improper ejection of fluid from a device, and features that add convenience or efficiency such as trigger mechanisms, systems and methods that allow for multiple types of fluid delivery or multiple ejections of multiple volumes of the same or different fluid, methods of controlling or programming volumes or penetration depths of an injection or instillation, or other features of one or multiple fluid deliveries.

Devices, systems, and methods are provided that allow for injection of a therapeutic fluid. The devices may be used for various applications related to conditions of a lower urinary tract, or nearby tissue, such as the urethra, bladder, kidney, ureters, prostate, etc. In particular embodiments, a fluid such as a pharmaceutical or other active chemical or biological agent can be injected into tissue of the urethra, prostate, bladder, or bladder neck The devices are designed to place one or multiple fluid delivery orifices at a desired location within the lower urinary tract, kidney, ureter, etc., to allow delivery (e.g., injection or instillation) of therapeutic fluid to desired tissue.

The invention identifies and addresses certain practical problems associated with other modes of delivering fluid to tissue of the lower urinary tract. For example, injection of fluid to the bladder by use of a single needle at a distal end of a rigid shaft can require specialized dexterity and experience of a doctor due to the cumbersome nature of a rigid shaft, with just one needle. Fluid delivery devices and methods as described herein are advantageous compared to the use of a single needle at a distal end of a rigid shaft, for various reasons including optional flexibility of a shaft, the use of multiple needles or needleless fluid delivery orifices, and the ability to locate, move, extend, open, or close multiple needles or fluid delivery orifices as desired to eject fluid for injection of the fluid into a tissue or instillation of the fluid at a tissue surface.

Devices and methods include various features discussed herein, any of which can be used either separately or in combination with any one or more of the other described features. Exemplary features include the following: construction of a shaft of the device in multiple, separable pieces, or as a single “integral” piece; a rigid shaft or a flexible shaft; the ability to move, extend, open, and close fluid delivery orifices; the ability to deliver multiple volumes or different types of fluid; multiple fluid delivery systems (e.g., orifices and reservoirs); features relating to the number and positioning of fluid delivery orifices such as multiple extendible needles or multiple extendable needleless fluid delivery orifices located at different positions along a length of a shaft of a device or located at different positions around a perimeter of a shaft of the device, and moveable fluid delivery orifices that may be moveable along a length of a shaft, around a perimeter of a shaft, along a length and around a perimeter of the shaft, or to extend distally from the end of a shaft or laterally or radially from a shaft; locating mechanisms such as balloons or other mechanisms to fix the location of a portion of a device, e.g., within the urethra, bladder, or bladder neck; and safety features that prevent inadvertent or improper actuation of a device or ejection of fluid from a device; and others described herein.

Devices of the invention include one or more fluid delivery orifices that are in fluid communication with one or more fluid reservoirs. A fluid reservoir includes an amount of fluid of sufficient volume to allow one or multiple ejections of fluid to be delivered from one or multiple fluid delivery orifices at the distal end of the device. A lumen may connect a fluid delivery orifice to a fluid reservoir, which may be located at a proximal or distal end of the device, or remote from a proximal end of a device in a console. One or multiple fluid delivery lumens may connect a fluid delivery orifice to a fluid reservoir located at any portion of the device, e.g., at a location at a distal end of the shaft, at a location at a proximal end of the shaft, at a body located at the proximal end of the shaft, or remote from the shaft and the body, such as at a remote console.

Generally, one exemplary type of a fluid delivery device can include a body at a proximal end. The body can include a handle that allows a user to grasp and manipulate the device, e.g., to insert the device shaft by manipulation of the body. A body can also include actuating features, e.g., for steering a steerable distal end of a steerable shaft, to actuate a fluid delivery, to move a moveable or extendable fluid delivery orifice, optional ports to connect the body to a remote console, and optional optic features such as a lens to allow viewing through an optical feature (to view a location of delivery).

A body may optionally include one or more fluid chambers (e.g., reservoirs) and a mechanism to apply pressure to the fluid. A shaft is attached to the body. A fluid chamber can be a fluid reservoir, a syringe chamber, or a device may include both, at the proximal end or at the distal end. A reservoir can refer to a fixed-volume holding space for fluid and need not (but may) be capable of being pressurized to low or moderate pressure or highly pressurized, e.g., pressurized to allow for priming or to cause fluid to be ejected from a needle or a needleless fluid delivery orifice, e.g., by way of a fluid delivery lumen. The pressure may be sufficient to cause the fluid to penetrate tissue, or to be applied to a surface of a tissue without penetration. A reservoir can be sized to contain one or multiple volumes of fluid, and may be in the form of a removable or replaceable vial.

Another exemplary type of fluid chamber or reservoir is referred to as a syringe chamber, which is a chamber that has a variable volume based, e.g., on a plunger, piston, bellows, or other mechanism for increasing or decreasing the volume (and pressure) of the chamber. A syringe chamber can be pressurized by a pressure source attached to the plunger, bellows, or piston such that fluid contained in the syringe chamber is ejected under pressure from the syringe chamber, e.g., for priming a device, for ejecting fluid from a delivery orifice to cause installation of a fluid to contact and cover a tissue surface of a tissue without penetrating the tissue, or for ejecting the fluid with a pressure to inject the fluid by penetration of tissue. The pressure source may be any source of energy (e.g., mechanical, electrical, etc.) such as a spring, solenoid, compressed air, manual syringe, electric power, hydraulic, pneumatic pressure sources, etc.

Attached to the body, an exemplary fluid delivery device can include an elongate shaft for insertion into the urethra, e.g., through the meatus or through a perineal incision, preferably through the meatus. Advantageously, the ability to install a device through the meatus instead of an external incision may allow a patient to be treated on an out-patient basis. The use of a flexible shaft provides for improved patient comfort.

The device includes a distal end and a proximal end. A distal end, including a shaft, generally is considered to include the portion of the device that is located internally within a patient's body during a treatment procedure. A distal end will typically include functional features that operate on fluid or tissue during use, such as one or more fluid delivery orifices, a delivery head or extension (“orifice extension”) that supports or contains one or more delivery orifice, one or more balloons or other forms of positioning devices or locating mechanisms if used, optionally a drainage orifice connected to a drainage lumen, optional opening and closing mechanisms to allow access to needleless fluid delivery orifices, optics, etc. A distal end may also include fluid delivery means, e.g., a fluid reservoir, a pressurizing mechanism, a fluid delivery lumen connecting a fluid reservoir to a fluid delivery orifice, or two or more of these in any combination.

An orifice extension can be any structure that can be manipulated and controlled to move a fluid delivery orifice a distance from the shaft. One example is a mechanical paddle that can be actuated to extend away from and retract back toward the shaft, and that includes a fluid delivery orifice on the paddle such as at the moveable end of the paddle, with the other end of the paddle being located at the shaft. Another example of an orifice extension is an extendable lumen that can be extended and retracted from a location along the length of the shaft or at the end (“tip”) of the shaft. Yet another example of an orifice extension is a balloon, fan, or other membranous structure that includes one or multiple fluid delivery orifices at a location on the balloon, and a lumen (e.g., an extendable lumen) connecting the fluid delivery orifice to the shaft. The balloon, fan, or membrane can be expanded, splayed, or unfolded, to cause the fluid delivery orifice to be moved along with the balloon, fan, or membrane, away from the shaft.

Regarding orifice extensions that include or that are in the form of an extendable lumen, an “extendable lumen” is a lumen that can be extended from the shaft. An extendable lumen may be moved to extend an orifice of the lumen a distance from the shaft (i.e., place the orifice away from the shaft) in a direction that includes a component in a longitudinal direction along a longitudinal axis of the shaft, in a direction that includes a component lateral from the longitudinal axis of the shaft, or in a direction that includes components in both directions.

An extendable lumen can generally be any lumen capable of delivering fluid as described herein, and may necessarily be of suitable size and structure and mechanical properties to allow for desired movement, mechanical properties, and fluid delivery. A combination of reduced size, desired flexibility and elasticity, and strength and ability to withstand elevated fluid pressures, are useful. Features such as the material of the lumen, wall thickness, and inner and outer diameters, can combine to produce desired strength and flexibility. An inner diameter can preferably be large enough to reduce pressure drop, and a desired wall thickness and material can allow for desired pressure resistance and flexibility.

A lumen structure can exhibit continuous dimensions of inner diameter, outer diameter, and wall thickness, along an entire length of a lumen. Alternately, a lumen may change dimensions (e.g., wall thickness) along the length of the lumen, with a larger wall thickness (greater outer diameter) at a proximal end and a thinner wall thickness (reduced outer diameter) at the distal end. Exemplary dimensions for a lumen as discussed herein can be any that are suitable for a balance of properties including pressure drop, strength, and flexibility, depending on overall system design and whether a fluid is delivered by injection or instillation. A balance is necessary, for example, because a relatively smaller inner diameter can increase a pressure drop; a narrower lumen wall can increase flexibility but reduces strength. For a device that includes a remote console (see infra), for fluid injection (as compared to instillation), an example of an inner diameter can be greater than 0.020 inches, e.g., from 0.022 to 0.030 inches (for a lumen made of polyetheretherketone, or “PEEK,” see below); exemplary outer diameters for the same exemplary lumen may be at least 0.032 inches e.g., from 0.034 to 0.045 inches.

A lumen may be made of any material that is suitably flexible, elastic, and strong, such as a metal (e.g., nitinol, stainless steel, or other metals useful with medical devices); metal reinforced polymer; polymer composites; or polymeric materials. Exemplary lumens may be made of high strength polymer such as polyimide, polyetherimide available from General Electric under the trade name Ultem®, and linear aromatic polymers such as PEEK™ (polyetheretherketone) available from Victrex plc. In some embodiments, a non-metal, polymeric lumen can be reinforced through the inclusion of materials including nano-particles, clays or glass. In some presently contemplated embodiments, a non-metal, polymeric lumen can be reinforced with one or more polymers, carbon, graphite or glass fibers, such as, for example, tubes braided with Kevlar or other high-strength polymers. See, e.g., U.S. provisional patent application Ser. No. 60/866,741, filed Nov. 21, 2006, by Crank, entitled INJECTION TUBE FOR JET INJECTION DEVICE, the entirety of which is incorporated herein by reference.

A lumen can be fabricated so as to have a burst strength exceeding at least about 2,000 pounds per square inch (psi) and in some embodiments a burst strength within a range of about 2,000 psi to about 5,000 psi. The lumen can be fabricated so as to have distention properties, wherein an orifice or jet port located at a distal end of the lumen retains its shape and size without suffering swelling, which swelling could have a detrimental effect on a fluid jet used to deliver therapeutic fluid.

A proximal end of exemplary fluid delivery devices can include a body that remains external to the patient during use. A proximal end can include structure (e.g., a handle or other type of grip) to allow handling of the device, including manipulation of the shaft, during use of the device. The proximal end also can include other features such as those that are not required or not able to be internal during a treatment procedure. Examples of features that may be part of a proximal end, e.g., a body, include a source of fluid and a source of pressure for the fluid; an eye-piece of the type used with an endoscope, or other optical feature, if included with the device; mechanical features such as a trigger or handle for holding or actuating a lumen, lumen extension fluid delivery means, or another feature at the distal end; adapters for attaching the proximal end to appurtenant equipment such as a source of power, a source of pressure, a source of fluid, or a source of vacuum; a drainage port for connecting to a drainage lumen, etc.

A body may also, alternately, or in addition, include one or more attachment ports to attach the body to an external and optionally remote component such as an external or remote pressure source, vacuum source, or an external or remote fluid reservoir. For example, a body may have a fluid port that attaches remotely to a console that contains a source of a fluid. The console can include a fluid reservoir and a pressure source capable of pressurizing the fluid to flow from the console, through the body, through a fluid delivery lumen in the shaft, and then through a fluid delivery orifice.

A shaft of a fluid delivery device may be an elongate component that in general extends from a proximal end to a distal end and includes features and componentry that allow for use and operation of distal end features by use and operation of proximal end or other external features. A shaft may generally be of various constructions, as desired, e.g., may be of an integral construction that is not designed to be assembled and dis-assembled prior to, during, or after use; or may be of a multi-piece construction that includes multiple elongate shaft components or elements that fit together as an assembled whole for use in a surgical procedure and that can be assembled and dis-assembled before and after use if desired See, for example, US 2006/0129125 (the entirety of which is incorporated herein by reference) for descriptions of different types of flexible, rigid, integral, or multi-piece shafts.

Either of a multiple-component or an integral construction-type shaft may be flexible, steerable, or rigid and any such type of shaft may include any of the features of devices described herein.

Metal or polymeric materials may be useful for any type of shaft or for a component of any type of shaft. Materials that can be particularly useful for a rigid, multi-piece shaft may include rigid polymeric materials such as a rigid plastic or a rigid metal material or a rigid ceramic material or a composite material. Specific examples include nitinol, polycarbonate, stainless steel, ABS, polyimide, polyetherimide, nylon, PEEK, and the like.

Materials useful for a flexible shaft or a flexible component of a multi-piece shaft can be relatively flexible polymeric materials such as polymeric materials known to be useful for catheter devices such as urethral catheters (e.g., Foley catheters). Specific examples of flexible polymeric materials include silicones, polyurethanes, block copolymers like polyether block amides, rubbers, latex, and the like.

Materials that may be useful for a steerable shaft can include some of the same materials listed above for a flexible shaft, and may include metals or rigid polymers (e.g., for hinges), rigid or flexible polymers for support or external layers, metal-reinforced polymers, etc.

A single-component or “integral” shaft for a fluid delivery device is a shaft that is substantially or completely assembled at the time of manufacture of the device and that is not designed to be assembled or dis-assembled prior to or after use. The shaft may be flexible, steerable, or rigid and may be prepared from metal or polymeric materials or combinations of such materials. A flexible integral shaft includes a flexible elongate component that extends from a proximal to a distal end of the fluid delivery device, and that defines or includes necessary functional elements such as one or more needles or needleless fluid delivery orifices or apertures for such orifices, lumens, triggers, actuating mechanisms, steering mechanisms, hinges for a steerable shaft, optics, etc., to operate the features at the distal end from the proximal end or remotely. These features of an integral shaft can be substantially permanent features of the device that are not designed to be removed from or dis-assembled into multiple components of a shaft.

According to certain embodiments an integral shaft can be a flexible shaft prepared from a flexible polymer, and can include lumens (e.g., fluid delivery lumens, drainage lumens, inflation lumens, etc.), actuating mechanisms, optics such as fiber optics, and any other necessary mechanical features that connect distal end features or mechanisms to a proximal end of a device. The lumens can be flexible lumens defined by or embedded in the shaft or in an internal or external wall or surface of the shaft.

If necessary a lumen can be of sufficient strength to withstand operating pressures such as in the case of an fluid delivery lumen that connects a fluid delivery orifice at a distal end to a pressurized supply of fluid at a proximal end. Exemplary elevated pressures (“injection pressures”) may be 2000 pounds per square inch or greater. A fluid delivery lumen may be of a flexible material (e.g., a metal or polymeric tube) that can withstand such an injection pressure, and may be prepared from exemplary materials capable of withstanding pressure of an injection, e.g., nitinol, stainless steel, reinforced (e.g., braided) polymer, as also described elsewhere herein.

A fluid delivery lumen can also be constructed and assembled to allow movement of a fluid delivery orifice as described herein. As an example a fluid delivery lumen may be capable of moving longitudinally along the length of the shaft to allow the fluid delivery lumen to be extended distally and optionally laterally from the distal end, e.g., the tip, of the shaft. Alternately or in addition, a fluid delivery lumen may exit a shaft at a position along the length of the shaft, exiting the side of the shaft, to allow lateral movement of the fluid delivery orifice away from a longitudinal axis of the shaft by bending or deflecting of fluid delivery orifice extension that moves relative to the shaft.

Actuating mechanisms may include or be in the form of mechanical or electronic features or connections such as wires, hinges, levers, or other connections and mechanical devices between a proximal end and a distal end, used to operate the device, such as to extend needles (or other fluid delivery orifice extensions) as desired, to actuate a syringe or pressure source at the distal end, to move fluid delivery orifices or orifice extensions that include fluid delivery orifices, etc. Other examples of actuating mechanisms at a proximal end may include plungers or other actuators useful to cause injectate to flow, under pressure, through a fluid delivery lumen and to a needle or a needleless fluid delivery orifice, to instill or injection to tissue.

The term “flexible shaft” refers to a shaft that is sufficiently pliable to allow bending and flexing that allow the shaft to be inserted through the meatus or an external incision, into the urethra, and to allow a portion of a distal end of the shaft to be guided into the urethra and optionally the bladder neck or bladder, as can be done with a Foley catheter. A flexible shaft can be sufficiently soft and pliable to conform or partially conform to a patient's anatomy, such as would a Foley-type catheter. In contrast, a “rigid” shaft is substantially rigid, which allows for multi-piece construction and for control of the distal end by manipulation of the proximal end, during use. A rigid shaft can typically be a metal or similarly rigid type of shaft as used with, e.g., types of rigid endoscopic or laporoscopic surgical devices. A “steerable” shaft is non-rigid and is flexible, so for purposes of this description a steerable shaft is considered to be “flexible,” but may be relatively less flexible than a shaft of the type typical of a Foley catheter due to a construction that includes a steering mechanism. A “steerable” shaft can be of the type used for steerable endoscopes or other steerable medical devices.

An integral shaft such as for a steerable or a flexible device shaft can be constructed with materials and methods similar to those used to prepare known urethral catheter devices such as a Foley catheter, a steerable endoscopic device, etc., but adapted to allow the device to include one or multiple fluid delivery orifices (e.g., a needle or needleless fluid delivery orifice) and optionally other features as described herein. An integral shaft may include any one or more of: one or multiple (e.g., flexible) fluid delivery orifices; moveable fluid delivery orifices that can be moved along a length or perimeter of a shaft; orifice extensions (such as a mechanical extension (e.g., paddle), a balloon, a needle, or an extendable lumen, etc.) that allow extension of a fluid delivery orifices laterally or longitudinally away from a shaft; tissue tensioners; balloons for locating the device; an optical feature such as a flexible fiber optic cable; ports or other opening and closing mechanisms for selectively exposing a needleless fluid delivery orifice to allow ejection (for injection or instillation) of a fluid from any one or other of multiple fluid delivery orifices; one or multiple lumen extensions that each include one or multiple fluid delivery orifices (i.e., in orifices parallel or in series); an array of lumen extensions that are separately or cooperatively extendable from a shaft; etc. Any one or more of these features can be located along a desired length of a device or at a location at a distal end of a device, along the integral shaft, and can be functionally connected to the proximal end by lumens or actuating mechanisms.

An integral shaft can be particularly useful with devices that include a flexible shaft and that contain a device-locating mechanism such as one or more balloons at the distal end for locating the device during use. The use of a device-locating mechanism may advantageously eliminate the need for an optical feature such as an endoscope, allowing for fluid deliveries to be carried out with blind vision methods. (Still, other embodiments of devices include both a flexible (e.g., steerable) shaft in combination with an optical feature.) Particular devices of the invention may include an integral flexible shaft; multiple needles or needleless fluid delivery orifices for making multiple fluid deliveries from the distal end of the device, which may be optionally moveable along a length or perimeter of a shaft, or extendable from a shaft; multiple fluid delivery systems to allow delivery of multiple different fluids; and other features of a urethral catheter such as one or more inflatable balloons at the distal end, and a drainage orifice at the tip and a drainage lumen leading from the bladder (when installed) to the proximal end of the device. Such devices, including a flexible shaft, a drainage lumen to drain urine from a bladder (e.g., as does a Foley catheter), and optional balloon, may be referred to as “fluid delivery catheter” embodiments of devices the invention. In use, “fluid delivery catheter” embodiments may be inserted through the external orifice of the urethra (meatus) as with a Foley catheter, as opposed to being inserted through an external incision and a tissue path to the urethra as with rigid-shaft fluid delivery devices.

An integral shaft can also be particularly useful with devices that include a flexible, steerable shaft and that contain an optical mechanism (including a light to illuminate the location of delivery) that allows the user to view a location of fluid delivery and to steer the distal end or tip of the device as desired at the location of delivery, to allow accurate placement of fluid delivery. Particular devices of the invention may include an integral steerable shaft; one or multiple needles or needleless fluid delivery orifices for making multiple fluid deliveries from the distal end of the device (of the same or multiple different fluids); the fluid delivery orifices can optionally and preferably be moveable along a length or perimeter of the device, or extendable longitudinally, distally, or laterally, away from the shaft The device can optionally include other features of a urethral catheter such and a drainage orifice at the distal end and a drainage lumen leading from the bladder (when installed) to the proximal end of the device. In used, these device embodiments, including a steerable shaft, a drainage lumen, and optical feature, may be inserted through the external orifice of the urethra (meatus) as with a Foley catheter, as opposed to being inserted through an external incision and a tissue path to the urethra as with rigid-shaft fluid delivery devices.

As opposed to an integral shaft, shafts referred to as “multiple-component” shafts may include two or more elongate pieces or components that fit together as an assembled whole and that can be dis-assembled prior to or after use. Typical components of a multiple-component shaft include an outer shaft or “sheath” in the form of an elongate rigid hollow sheath, and one or more additional inner shaft components that can be assembled together with the outer shaft to form a functional, assembled, multi-component shaft.

An outer shaft or “sheath” of a multiple-component shaft may be a basic sheath that is sized and shaped to be placed in the urethra while containing one or more inner shaft components. A sheath component of a multi-piece shaft may include just a hollow and rigid sheath, or may include a hollow and rigid sheath having functional features of a device such as multiple needles or needleless fluid delivery orifices; one or more tissue tensioner; a fluid delivery lumen or delivery head; a positioning component such as one or more balloons; and one or more lumens that connect a functional feature at the distal end of the sheath to the proximal end. An exemplary outer shaft can be a rigid (e.g., of metal or a rigid plastic) sleeve that can be inserted in a patient through the urethra or through an external incision at the perineal region. When inserted, one or more inner shaft components can be inserted into the outer shaft, as desired.

An inner shaft component of a multi-component shaft can fit within the outer shaft component or sheath and may include one or multiple functional features of a device such as needles or needleless fluid delivery orifices; one or more lumens; one or more delivery heads; or an optical feature such as a lens, open viewing channel; a tissue tensioner; etc. An inner shaft that includes needles or needleless fluid delivery orifices may be specifically referred to as, e.g., an inner “fluid shaft,” and may include one or multiple needles or needleless fluid delivery orifices at a distal end connected through one or multiple fluid delivery lumens to a proximal end of the inner shaft component. Optionally, an inner shaft component (or a feature thereof such as a needle, a fluid delivery orifice, or a delivery head) can be moveable within an outer shaft component or shaft generally, for any reason, such as to allow movement of a delivery head or fluid delivery orifice during use.

Also optionally, as desired, embodiments of the invention may combine multiple device features into a single rigid shaft component. For example, a rigid shaft component may combine an outer sheath with an inner fluid shaft, and may be sized and shaped to receive an endoscope. Alternately, a rigid shaft-component may combine an outer rigid sheath with an endoscope and may be sized and shaped to receive an inner “fluid shaft” that contains a fluid delivery lumen and fluid delivery orifice. As yet another alternative, the endoscope may also be combined with an outer shaft component and injection features into a single rigid shaft that is not designed to be assembled and disassembled.

Generally, any of the various shaft designs may be used with either blind vision methods or direct vision methods. An optical feature can be incorporated into any of a multiple-component, integral, rigid, steerable, or flexible shaft, using known materials and constructions, such as with an optical feature of the type known to be useful for endoscope type devices, including any form of fiber optic device or other optical device. An eyepiece can be located at the proximal end of a device and one or more of an open vision channel, optical fiber, lens, multiple lenses, mirrors, refractive or reflective devices, or combination of these, can be used to create visual communication between the proximal end and a location at the distal end of the device. For a flexible (e.g., steerable) shaft, for example, a flexible optical fiber can run from an eyepiece at the proximal end of the device to the distal end of the device at a location along the shaft The optical fiber allows viewing of the distal end of the shaft, e.g., at a location to view a fluid delivery orifice.

With direct vision methods, an optical feature such as an optical fiber can be used to view internal tissue such as the internal urethra, bladder, bladder neck, ureter, or kidney, and, optionally in combination with a steerable shaft distal end, a direct vision method can facilitate accurate placement of a fluid delivery orifice or multiple fluid delivery orifices as desired, e.g., within the prostatic bladder, bladder neck, bladder, ureter, kidney, etc.

Blind vision methods, on the other hand, can eliminate the need for an optical feature and may instead rely on positioning of injection needles or needleless fluid delivery orifices by use of other features such as the known dimensions of a device or positioning features at the distal end of the device, e.g., one or more of a distal or a proximal balloon, or other locating mechanism, distance demarcations at the proximal end of the device, tissue tensioner, moveable orifice, which together can allow for blind fluid delivery at a desired tissue location.

Any combination of shaft properties (e.g., rigid or flexible (e.g., steerable) shaft) and other features described herein can be useful in fluid delivery devices, as desired. Certain specific features or combinations of features may be particularly useful with either rigid or flexible shaft designs. A rigid multi-component shaft or a steerable integral shaft may be particularly useful in combination with a direct vision feature and may optionally exclude the use of other types of positioning features such as one or more balloons at the distal end of the device.

Certain integral, flexible shaft embodiments of devices may be useful as including a flexible shaft and can provide advantages such as patient comfort due to the flexible shaft, elimination of the need for an external incision to access the urethra (as is normally used with rigid shaft designs) and optionally the use of blind vision fluid delivery methods based on the use of positioning features such as moveable or extendable needles or needleless fluid delivery orifices that extend laterally or distally from the shaft, or injection balloons at the distal end of the device. Flexible-shaft devices may include a flexible shaft that includes features for placing and fixing the distal end of the device to locate one or more needles or needleless fluid delivery orifices as desired, e.g., within the bladder (i.e., “positioning features” or “focusing features”). Advantageously, the flexible shaft may be inserted through the external urethra orifice (meatus) (e.g., in the manner of insertion used for a Foley catheter) without requiring an external incision or a tissue path from the external incision to the urethra. The use of positioning features can avoid the need for an optical component for locating a distal end (although other flexible shaft embodiments advantageously can include an optical feature for direct viewing). Exemplary non-optical positioning features include one or a combination of: visible distance demarcations at the proximal end that can be used to gauge the location of a distal end, a fluid delivery orifice, or positioning feature (e.g., balloon or other structure) relative to the bladder or bladder neck or bladder; known dimensions of the device; one or more balloons; needles or needless fluid delivery orifices that extend from the shaft within the bladder to allow placement as desired within the bladder; or other positioning features at the distal end of the shaft. Also useful to avoid the need for an optical feature are multiple or moveable features such as multiple or moveable (e.g., extendable) fluid delivery orifices. Multiple fluid delivery orifices can be located at multiple positions along a length or perimeter of the distal end of a device and may be independently used to deliver fluid at any of the multiple locations of the needles or needleless fluid delivery orifices. Moveable needles or needleless fluid delivery orifices allow movement of a needle or needleless fluid delivery orifice along a length or perimeter of the device after the device may be to some degree fixed internally. Alternately, an extendable fluid delivery orifice may extend distally from the distal end or the tip of the device shaft, may extend laterally from along the shaft, or may extend laterally and partially in a direction either proximally and distally (longitudinally) along the shaft.

According to embodiments of devices of the invention, multiple needles or needleless fluid delivery orifices (collectively, “fluid delivery orifices”) are located at the distal end of the device at a location or locations along the shaft that place the needles or needleless fluid delivery orifices at a desired location internal to the patient upon installation, e.g., within the bladder (which includes the bladder neck), or other location of the lower urinary tract. The needles or needleless fluid delivery orifices are in fluid communication (e.g., through an fluid delivery lumen) with a fluid source (one or multiple fluid sources for multiple orifices) that can be pressurized to eject fluid from the fluid delivery orifice to inject fluid into tissue or to instill or coat a surface of an internal tissue. The multiple orifices may move together or separately along the length of the device, distally, or laterally, as described. Each needle or needleless fluid delivery orifice can connect to a mechanism at the proximal end of the device to allow movement or extension of the orifice, with multiple orifices being moveable or extendable together or independently. Each orifice may also be in fluid communication with a fluid source (e.g., at a proximal end or a distal end of the device) and with an activating mechanism at the proximal end to cause ejection of a fluid from the orifice, to allow fluid to be ejected from each orifice separately or in combination.

A fluid delivery orifice may have any useful size (e.g., length and diameter) for producing desired properties of an injection such as desired exit velocity, fluid volume, fluid dispersion (e.g., size and shape of a cloud of injected particles), etc. Examples of useful orifice diameters may be in the range from about 0.001 to 0.05 inches, depending on factors such as whether fluid is being injected to penetrate tissue or instilled to contact tissue surface, and if injected, desired injection parameters and the type and size (e.g., depth) of tissue being injected. The fluid delivery orifice may be larger or smaller than the fluid delivery lumen adjacent to the fluid delivery orifice, if desired, to affect the exit velocity of the fluid at the fluid delivery orifice. Examples of useful orifice shapes may include features such as a venturi, a continuous uniform diameter along the length of an orifice, a funnel-shape, etc. Often, a relatively smaller diameter orifice may produce an injection depth of greater penetration into tissue compared to a larger diameter orifice (with identical injection pressure). In general, a size and shape of injection orifice can be chosen for the particular drug delivery task (injection, instillation, etc) and tissue characteristics.

The pressure source for pressurizing a fluid for ejection from a fluid delivery orifice may be mechanical (such as a spring or a solenoid), pneumatic, pressurized gas such as carbon dioxide, hydraulic, electric, etc., as will be understood, and may be located at the proximal end, the distal end, or along the shaft. The pressure source may be mechanically or electronically controlled. The pressure source can cause a fluid contained in a fluid reservoir (e.g., a fixed- or variable-volume chamber) to be pressurized to a transient pressure, at a fluid delivery orifice, that is sufficiently high to produce desired amount of flow of fluid from the fluid delivery orifice with sufficient force to either coat tissue or penetrate into tissue.

Additional optional features of fluid delivery devices of the invention, for optional use in combination with other features described herein, include inflatable balloons located at the distal end of the shaft of the device, e.g., as “positioning features.” A fluid delivery device may include one or multiple balloons at a distal end, allowing the device to be placed and fixed at a desired position during use. A balloon for locating the fluid delivery device can be particularly useful in combination with a device that does not include any optical feature such as an endoscope to directly view the operation of distal features of the device such as a fluid delivery orifice. Placement of a balloon at the distal end of the device, at a known distance from a fluid delivery orifice, can facilitate proper placement of the fluid delivery orifice, such as at the bladder neck, based on the positioning of the balloon.

An example of a balloon that allows a surgeon to locate a fluid delivery device as desired is a balloon that can be placed within the bladder or at the bladder neck when the device is installed. The balloon may be of a type used in a Foley catheter but adapted to function at the end of rigid or flexible shaft of a fluid delivery device as described herein. The balloon can be useful to fix the overall location of the device during use, i.e., to place the shaft of the device at a desired location to cause one or more fluid delivery orifices to be located as desired, e.g., within the bladder or bladder neck. The balloon can be located on the shaft at a location distal to or proximal to fluid delivery orifices. When the device is installed, the balloon can be in the bladder or bladder neck, and can properly locate the device during treatment, and can also seal the bladder neck from the bladder or bladder neck.

Another feature of a fluid delivery device for optional use in combination with other features described herein, is a tissue tensioner located at a distal end of the device. A tissue tensioner can be located at the shaft, somewhat near to a fluid delivery orifice, e.g., to be within the urethra or bladder and near the fluid delivery orifice when the device is installed. A tissue tensioner can be a mechanism capable of contacting tissue, e.g., urethral or bladder tissue, to hold a desired portion of the tissue in place relative to a fluid delivery orifice, and to optionally produce a tension or strain on the tissue in a manner that can affect the manner in which fluid penetrates the tissue and becomes distributed in the tissue upon injection. While a tissue tensioner can be used in combination with any of the other features described herein, including rigid shaft embodiments of devices, a tissue tensioner may be particularly useful when used with a device that includes a flexible shaft. The tissue tensioner can facilitate a good result upon injection of fluid through tissue of the lower urinary tract by ensuring that the tissue is fixed and includes a desired amount of tension for receiving an injection.

Examples of types of tissue tensioners include inflatable balloons located at a shaft near a fluid delivery orifice, and mechanically extendable or retractable components such as paddles, protrusions, levers, metal cages, and the like, any of which can be extended from a shaft of a fluid delivery device to place pressure on internal tissue, e.g., on urethral tissue within the bladder or bladder neck. Tissue tensioners are described in Assignee's copending patent application U.S. Ser. No. 11/186,218, entitled “NEEDLELESS DELIVERY SYSTEMS,” filed Jul. 21, 2005, the entire disclosure of which is incorporated herein by reference.

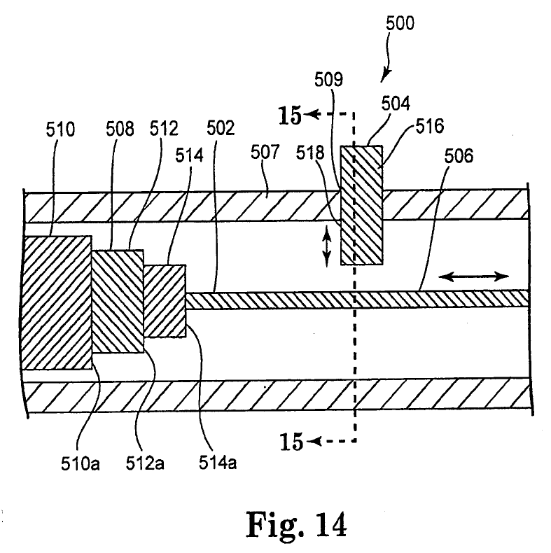

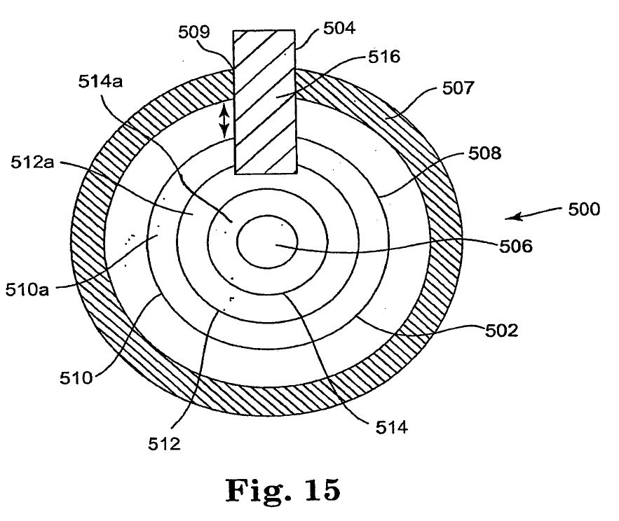

Another example of an optional feature that can be used with any of the devices identified herein, is a mechanical volume control feature as described in Assignee's copending U.S. Ser. No. 60/856,035, filed Nov. 9, 2006, by Crank et al., entitled MECHANICAL VOLUME CONTROL FOR INJECTION DEVICE, the entirety of which is incorporated herein by reference. An adjustable volume control feature can allow for adjustably controlling the delivery of fluid, when included in a device as described herein. The adjustable volume control generally includes a mechanical stop system with a plunger member and a stop member, wherein the plunger member and stop member physically interact to restrict a plunger insertion length, which simultaneously controls an amount of therapeutic fluid expelled by the plunger. In some embodiments, the stop member can be configured so as to be actuated coaxially with plunger movement while in other embodiments the stop member may be actuated transversely to the plunger movement An exemplary design for a mechanical stop system is illustrated at FIGS. 14 and 15.

A device can include a locating mechanism or locating mechanism that allows a user to place a distal end at a desired location in the lower urinary tract. An example can be a balloon, paddle, or other extension or extendable feature located at a distal end of a device, that can be extended or expanded to allow the structure to contact a bladder neck when the structure is located within the bladder. In use, a locating mechanism can be retracted within or against a shaft during insertion of a distal end of a device into the urethra, bladder, and bladder neck. Once the distal end is inserted to locate the locating mechanism within the bladder or bladder neck, the locating mechanism can be extended away from the shaft and the device (including the shaft and locating mechanism) can be moved (e.g., pulled) proximally by the operator. Pulling the locating mechanism proximally causes the locating mechanism to contact the bladder neck; the operator feels this resistance at the bladder neck and recognizes that the locating mechanism is in contact with the bladder neck and is in a desired location.

A device of the invention can optionally include multiple fluid delivery orifices at the distal end, some or all of which may be extendable or not Each fluid delivery orifice may be independently actuated for movement or ejection of fluid. Embodiments of multiple fluid delivery orifices may be in communication with each other and in communication with a common fluid source. In alternate embodiments, two or more fluid delivery orifices may each be in communication with separate fluid sources to allow delivery of multiple different fluids at the distal end of the device; i.e., a single device can include multiple fluid delivery systems (e.g., a single fluid delivery system can include a fluid delivery orifice in communication with a fluid delivery reservoir). Multiple fluid delivery systems can allow for delivery of two or more different fluids from a distal end of a single device. Each fluid may be different or the same, and may be delivered using the same or different parameters, such a delivery volume, delivery pressure, delivery to inject, delivery to instill, deliver to different tissues, etc. As an example, a device may delivery two different drugs to tissue of a urinary tract; optionally one drug may be applied to a surface of a tissue and one may be applied to tissue interior (i.e., injected). Alternately, two different fluids may be delivered to different tissue locations, e.g., two different locations of tissue around a circumference of a urethra or different locations along a length of a urethra. Two or more fluids may be delivered at the same pressure, at different pressures, at the same delivered volume, at different delivered volumes, etc.

The delivery of multiple different fluids can involve a device that includes more than one fluid reservoir, e.g., multiple fluid reservoirs that are independently capable of containing a supply of fluid for delivery, each able to delivery a different fluid through a different fluid delivery orifice. One or more reservoirs may be located at a distal end of a device, at a proximal end of a device, at a remote console, or at any combination of a distal end, proximal end, or remote console.

As used herein a distal fluid reservoir can be a volume at a distal end of a device shaft that can contain a fluid, that is of a volume greater than a volume necessary for a typical fluid delivery lumen. An example of a volume for a distal fluid reservoir may be a volume of a magnitude comparable to a volume of a fluid injection. A distal fluid reservoir is typically not necessary but may be desirable to improve movement of a fluid. For example a distal fluid reservoir can place an amount of fluid at a distal end of a device, proximal to a delivery orifice, which can reduce the need for movement of the fluid along the length of the device during injection or ejection. A reduced amount of movement of the fluid can result in more efficient fluid delivery.

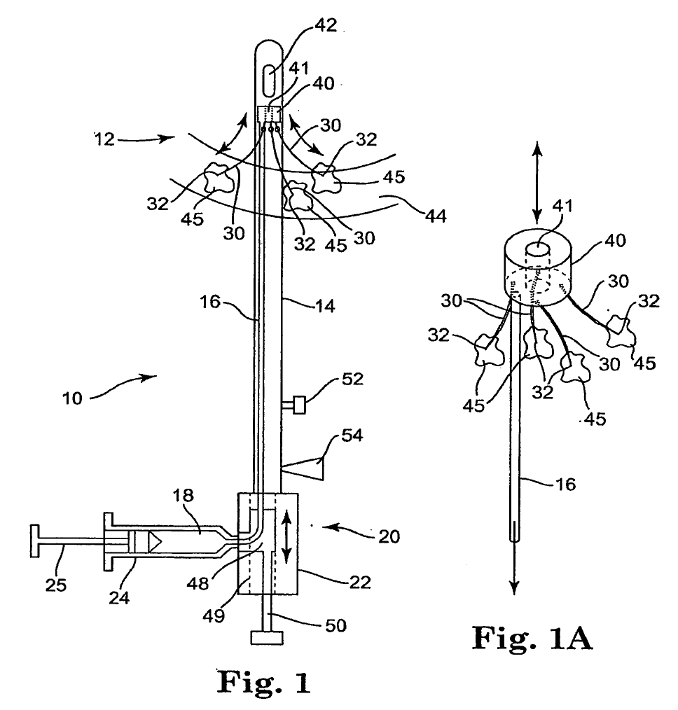

FIG. 1 illustrates an example of a device according to the invention, a fluid delivery device capable of blind delivery of fluid to the bladder or bladder neck. Device 10 generally includes distal end 12; proximal end 20, including body 22 and attached pressure source (syringe) 24; and shaft 14. Shaft 14 includes moveable fluid delivery lumen 16, which connects fluid reservoir 18 of syringe 24, at proximal end 20, to needles 30 and fluid delivery orifices 32. Needles 30 are in fluid communication with moveable distal fluid reservoir 40, and moveable distal fluid reservoir 40 is further in communication with moveable fluid delivery lumen 16 leading to proximal end 20, body 22, and reservoir 18 of syringe 24. Needles 30 and moveable fluid reservoir 40, when device 10 is installed, are located within the bladder, as represented by bladder tissue 44. As illustrated in this exemplary embodiment, a single fluid delivery lumen (16) connects a single proximal fluid reservoir (18) to a single distal fluid reservoir (40), and multiple fluid delivery orifices (32), allowing for a single type of fluid (45) to be delivered from multiple injection orifices at distal end 12.

Drainage orifice 42 allows for drainage of urine from the bladder during use, and is connected to proximal end 20 through a drainage lumen (not shown) in shaft 14. For example, a drainage lumen may be located at the interior of shaft 16, optionally extending through channel 41 of distal fluid reservoir 40.

Device 10 includes distal end 12, which includes multiple delivery orifices 32 located at ends of extendable needles 30. Extendable needles 30 can be extended and retracted from shaft 14 (e.g., through apertures in shaft 14 and moving in the direction of arrows at distal end 12 in FIG. 1) by combined movement as an assembly of moveable fluid deliver lumen 16, moveable fluid reservoir 40, and needles 30, in a longitudinal direction along shaft 16 (longitudinal movement is illustrated by arrows on body 22 of FIG. 1, and by the arrows of FIG. 1A). Movement of the assembly of needles 30, reservoir 40, and lumen 16, can be accomplished by movement of a mechanism built into body 22. As illustrated in FIG. 1, that mechanism includes a simple sliding chamber 48 that connects syringe 24 to moveable fluid delivery lumen 16, and that moves relative to shaft 14 within open channel 49 (shown in dashed lines) of body 22, by movement of slidable handle 50. Slidable chamber 48 can be moved together with syringe 24, relative to body 22 and shaft 14.

Extendable needles 30 can be placed in a retracted position to allow for insertion of distal end 12 into a patient, transurethrally, through the meatus, to locate needles 30 within the bladder, e.g., near the bladder neck. An optional balloon (not shown at figure) at the distal end can be inflated inside of the bladder. Once installed, needles 30 can be extended from the shaft, extending laterally and proximally (toward the proximal end of device 12), to contact tissue 44 of the bladder neck or bladder. Each extendable needle 30 can be connected through fluid lumen 16 to one or more fluid reservoirs (e.g., reservoirs 18 or 40), at the proximal or distal end, or both, and injectate fluid can be pressurized to travel through fluid lumen 16 to extendable needles 30 for delivery at the bladder or bladder neck, either to inject or to contact tissue of the bladder neck FIG. 1 shows injectate fluid 45 that has been injected to penetrate tissue 44. The pressurization mechanism is shown as mechanical syringe 24 at proximal end 20, but may alternately be any type of pressurizing mechanism.

Other features of device 10 of FIG. 1 include an optional balloon port 52 at proximal end 12, in communication with a balloon (not shown) at distal end 12. Balloon port 52 can be used to provide fluid to pressurize and inflate an optional balloon when installed within the bladder. Optional drainage outlet 54 is illustrated at proximal end 20, which connects through a drainage lumen (not shown) to drainage orifice 42 at distal end 12. A slidable handle or “trigger” 50 at the proximal end is mechanically engaged with the extendable needles 30, through moveable lumen 16; the trigger 50 can be moved to move needles 30 and cause needles 30 to extend or retract from shaft 14. In the illustrated embodiment, trigger 50 can be actuated independently of syringe 24 so trigger 50 can be used to extend or retract needles 30 and syringe 24 can be separately actuated to eject fluid from needles 30.

Offset FIG. 1A illustrates an internal mechanism of the device, which is an assembly that includes multiple needles 30 in communication with hollow distal fluid reservoir 40. The assembly is moveable as a single assembly within the device, and when moved proximally (in the direction of the arrow at lumen 16) needles 30 extend proximally and laterally from shaft 14. Distal reservoir 40 connects at a proximal side to a fluid delivery lumen (16) that extends through a length of shaft 14 of device 12 to moveable syringe 24 at proximal end 20 of the device 12. Distal reservoir 40 also connects to multiple hollow needles 30 located on the proximal side of reservoir 40, extending toward proximal end 20 of device 10. As shown in FIG. 1, trigger 50 can be used to move the assembly of reservoir 40 and needles 30 (and moveable lumen 16) proximally to cause needles 30 to extend from shaft 14 at distal end 12 of device 10. Fluid within lumen 16, proximal reservoir 18, distal movable reservoir 40, and moveable needles 30, can be pressurized by use of plunger 25 of syringe 24 to cause the fluid to be ejected at once from each of orifices 32 of needles 30.

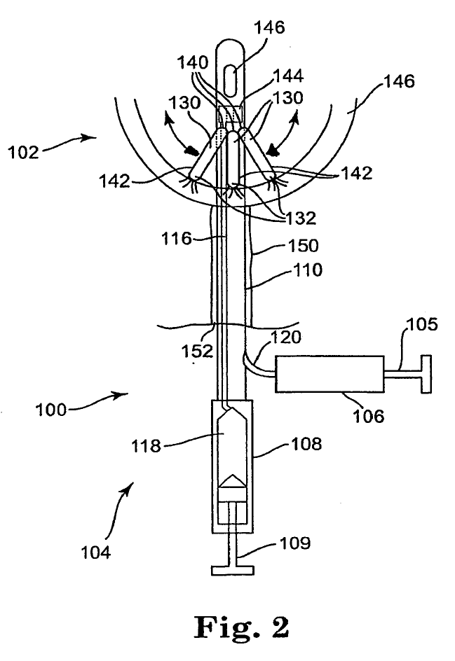

FIG. 2 illustrates a fluid delivery device for blind delivery of fluid to the bladder neck using a needleless delivery device. Device 100 generally includes distal end 102; proximal end 104, including body 106 and attached pressure source (syringe) 108; and shaft 110. Shaft 110 includes fluid delivery lumen 116 within its interior, which connects fluid reservoir 118 of syringe 108, at proximal end 104, to paddles 130 and fluid delivery orifices 132 at distal end 102. A first attached end 140 of each of paddles 130 is connected to shaft 110 at a hinged connection. A movable end 142 of each of paddles 130 includes a fluid delivery orifice 132. Each paddle 130 can move (as indicated by arrows) laterally away from shaft 110 by swinging on attached end 140, causing each fluid delivery orifice 132 at a moveable end 142 to extend away from shaft 110. Body 106, including plunger 105 connected to actuating mechanism 120 can be used to extend and retract paddles 130 away from and toward shaft 110 at their location at distal end 102 of device 100. Fluid delivery lumens (not shown) connect distal reservoir 144 to each paddle 130 to allow fluid to flow through a path connecting proximal reservoir 118 to each orifice 132.

Actuating mechanism 120 is attached at a proximal end to plunger 105 at body 106, and at a distal end to paddles 130, so that movement of plunger 105 causes moveable ends 142 of paddles 130 to extend and retract away from and toward shaft 110 (in the direction of the arrows). Paddles 130 are in fluid communication, through reservoir 144, with fluid reservoir 118, through fluid delivery lumen 116, extending through shaft 110.

As illustrated in this exemplary embodiment, a single fluid delivery lumen (116) connects a single proximal fluid reservoir (118) to a single distal fluid reservoir (140), and multiple fluid delivery orifices (132), allowing for a single type of fluid to be delivered from multiple injection orifices 132 at distal end 102.

During use of device 100, paddles 130 and distal fluid reservoir 144 are located within the bladder, as represented by bladder tissue 146. Shaft 110 is located within urethra 150 by entering at meatus 152. Drainage orifice 146 allows for drainage of urine from the bladder during use, and is connected to proximal end 104 through a drainage lumen (not shown) in shaft 110. Paddles 130 can function as a locating mechanism by being extended within a bladder and contacting tissue of the bladder or bladder neck upon movement of the device in a proximal direction by an operator.

Still referring to FIG. 2, distal end 102 includes multiple needleless fluid delivery orifices 132 at moveable ends 142 of each of paddles 130. Each paddle 130 may be extended away from the shaft laterally, hinged at the distal attached end 140 of each paddle 130 so the paddles 130 also extend with delivery orifices oriented to deliver a fluid in a proximal direction. The paddles 130 can be retracted against shaft 110 to allow insertion of device 100, e.g., through meatus 152, into a patient to locate paddles 130 within the bladder, e.g., near the bladder neck. Once installed, paddles 130 can be extended from shaft 10 as illustrated, extending laterally and proximally by rotating at attached (hinged) end 140, so moveable ends 142 of paddles 130 contact tissue 146 of a bladder or bladder neck, each paddle 130 can be connected separately or together, through one or more fluid delivery lumens 116, to proximal end 104 of device 100. An injectate fluid can be pressurized at a reservoir (e.g., 118) at proximal end 104 of device 100 and caused to travel through the fluid delivery lumen (116), optionally through a distal fluid reservoir (e.g., distal reservoir 144) at distal end 102, and to each of paddles 130 for delivery at the bladder or bladder neck through the needleless fluid delivery orifices 132 at the end of each paddle. The pressurization mechanism can be of any form, such as a plunger 109 of a syringe (108) controlled by a power source external to the device.

Other features of the device of FIG. 2 include a flexible shaft (shaft 110) that includes a drainage lumen (not shown) in communication with drainage orifice 146. An actuator (body 106 and plunger 105) to deploy paddles 130 is illustrated to be connected to shaft 110 at proximal end 104, and also connects mechanically to paddles 130 at distal end 102 to allow paddles 130 to be actuated to swing between extended and retracted positions. The actuator can be actuated independently of syringe 108, so the actuator can be used to extend or retract paddles 130, and syringe 108 can be separately actuated to eject fluid from fluid delivery orifices 132.

FIG. 3 illustrates another embodiment of a fluid delivery device (160) for blind delivery of fluid to the bladder or bladder neck (180) using a needleless delivery device. Distal end 164 includes multiple needleless fluid delivery orifices 168 at moveable end 182 of multiple paddles 166. Each paddle 166 may be extended away from shaft 190 laterally (shown by arrows) for example by movement of an internal assembly that includes paddles 166, and optionally pressure vessel (reservoir) 170 containing fluid injectate, plunger 174, and power source 172. Fluid delivery lumens connect distal reservoir 170 with each paddle 166. Depending on preference and application, device 160 can be used to deliver a single type of fluid through each orifice 168, together or independently. Or device 160 can deliver multiple different fluids, e.g., a different fluid from two or more of the paddles. Delivery of multiple fluids can be accomplished, for example, by including multiple distal reservoirs at distal end 164, one distal reservoir per fluid. Each distal reservoir can be actuated independently by a mechanism at proximal end 162.

In one embodiment, paddles 166 can be extended (together or independently) away from shaft 190 laterally and to point in a proximal direction by rotating at a hinged connection between shaft 190 and attached end 184. Paddles 166 can be retracted to lie against shaft 190 to allow insertion of distal end 164 of device 160 into a patient to locate paddles 166 within the bladder, e.g., near the bladder neck. The paddles can function as a locating mechanism. Once distal end 164 is installed, paddles 166 can be extended away from shaft 190 as illustrated, extending laterally, so moveable ends 182 of paddles 166 contact bladder tissue 180. Paddles can be extended (together or independently) by movement of actuating mechanism 176 at proximal end 162. Each paddle 166 can be connected separately or together to distal pressure vessel 170, also at distal end 164 of device 160. A fluid within pressure vessel 170 can be pressurized by movement of plunger 174 using power source 172 (e.g., pressurized carbon dioxide), which can be activated by actuator or “trigger” 175 at proximal end 162.

Other features of the device of FIG. 3 include a flexible shaft (190) and “trigger” (actuating mechanism 176) at proximal end 162, useful to deploy paddles 166. A trigger 175 to release injectate (fluid) is also illustrated at proximal end 162 of device 160, and connects mechanically or electronically (as indicated by the dashed lines) to power supply 172 to pressurize fluid in fluid reservoir 170 (by movement of plunger 174) to cause fluid to be ejected from needleless fluid delivery orifices 168 at moveable ends 182 of each paddle 166. The dashed lines between actuating mechanism 176, trigger 175, and distal end 164, indicates a mechanical or electronic connection between these elements of device 160 to the extent necessary to actuate distal end features from proximal and 162. As noted, multiple distal and proximal reservoirs connected by multiple fluid delivery lumens could be used to allow delivery of two or more types of fluid. The fluid may be pressurized to be injected to tissue, or to be ejected to contact tissue without penetrating the tissue (i.e., instilled at the surface of the tissue). As illustrated, fluid 192 is injected into and penetrates tissue 180.

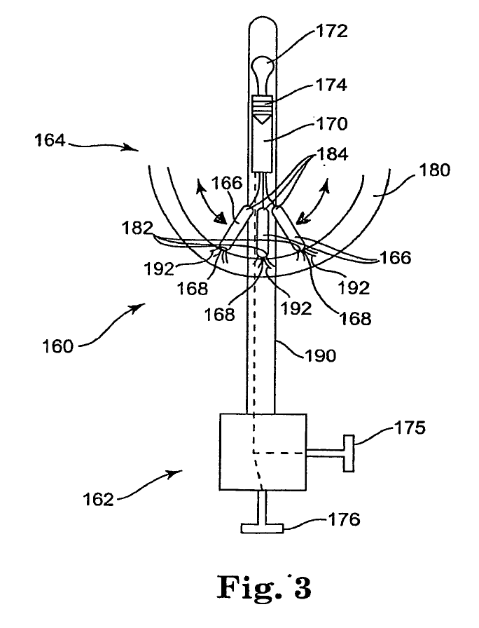

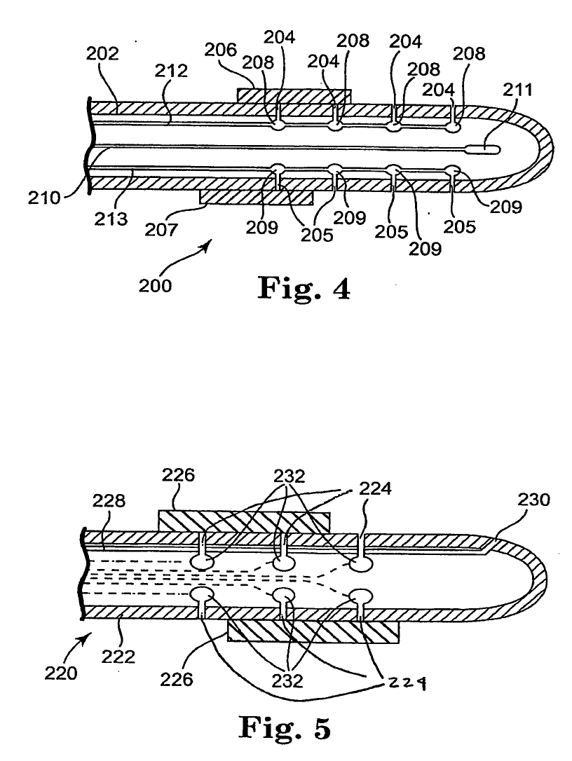

Referring to FIG. 4, illustrated is a distal end of a fluid delivery device. Distal end 200 includes shaft 202, sets of needleless delivery orifices 204 and 205, distal fluid reservoirs 208 and 209 (both are optional and could be replaced by a standard branched lumen), fluid delivery lumens 212 and 213, and moveable covers (e.g., sleeves) 206 and 207. Covers 206 and 207 are illustrated as sleeves, one sleeve each for each set of delivery orifices 204 and 205. Alternately, a cover could be any other type of single or multiple cover or covers that can allow for independent opening and closing of each single orifice or multiple orifice at once, such as any form of a moveable wall, door, flap, or other barrier, that can be moved to cover or uncover, or open or close, an orifice. For example, each of the illustrated four orifices 204 could have a separate cover that can be opened and closed independently. Similarly, each of the four illustrated orifices 205 could have a separate cover that can be opened and closed independently.

Fluid delivery orifices 204 are in communication with each other, through optional reservoirs 208, and with fluid delivery lumen 212; fluid delivery orifices 205 are in communication with each other, through optional reservoirs 209, and with fluid delivery lumen 213. Each set of orifices is associated with a moveable cover (206, 207); each cover is independently moveable and controllable (e.g., mechanically or electronically) from a proximal end of the device to allow separate control of covers 206 and 207 for covering and uncovering orifices 204 and 205, respectively. The exemplary device shows two separate fluid delivery lumens (212, 213), two sets of fluid delivery orifices (204, 205), two sets of optional reservoirs (208, 209), and two independently-operated sleeves (206, 207) that together allow for independent delivery of two different fluids, one through each fluid delivery lumen and set of orifices. More than two different types of fluid could be delivered from distal end 200, if desired, by addition of yet another (e.g., third, fourth, fifth, or more) fluid delivery lumen, orifices, optional reservoirs, and sleeve (e.g., cover).

FIG. 4 illustrates a delivery device distal end that includes two sets of needleless delivery orifices 204, 205, with the orifices of each set being “in series” (in communication with each other), and at different positions along a length of flexible device shaft 202. Each needleless delivery orifice 204 (205) is located near or behind a moveable cover (e.g., sleeve) 206 (207) that can be moved along a length of the shaft 202 to expose or cover (close) each orifice 204, either a single orifice 204 or 205 by itself, or in combination with one or more additional orifices (204 or 205). Only one cover is illustrated for each set of orifices 204 and 205, but more than one cover may be associated with each set of orifices, as desired, as an alternate method of allowing for independent opening and closing of each orifice.

Each delivery orifice can be connected to other orifices (e.g., orifices 204 are in fluid communication with each other, and orifices 205 are in fluid communication with each other but are not in fluid communication with orifices 204), and to a common fluid supply (e.g., a proximal fluid reservoir at a proximal end of the device, not shown). The fluid supply can be capable of being pressurized to cause fluid to be delivered from each connected delivery orifice that is not covered by a sheath; fluid can be delivered independently from any one of multiple delivery orifices connected to one fluid supply, or at once from two or more of multiple orifices connected to a common fluid supply.

Still referring to FIG. 4, covers (sleeves) 206 and 207 are illustrated at the external portion of shaft 202. Cover 206 is capable of covering one or more of fluid delivery orifices 204. Independently from sleeve 206, sleeve 207 is capable of covering one or more of fluid delivery orifices 205. As will be appreciated, each cover 206 and 207 can independently be mechanically actuated to move relative to shaft 202 in a manner to allow one or more of delivery orifices 204 or 205, respectively, to be covered or uncovered, as desired, to allow fluid to be delivered from any uncovered (open) delivery orifice 204 or 205, respectively. Distal end 200 of FIG. 4 is also illustrated to include a central drainage lumen 210 and a drainage aperture 211, which allow for drainage of the bladder when distal end 200 is installed in the patient.

Optionally, distal end 200 can include features such as a steerable shaft to allow end 200 to be steered (e.g., articulated) during installation and delivery of a fluid. Also optionally, distal end 200 can include an optical feature leading to a proximal end of the device to allow viewing of a delivery location during use and accurate placement of distal end 220, orifices 204 and 205, and fluid delivered through orifices 204 and 205.

FIG. 5 illustrates another embodiment of a portion of a distal end (220) of a fluid delivery device. The device may include a rigid or preferably a flexible shaft (222) and may be sized and designed for ejection of fluid at a location at the lower urinary tract or nearby tissue, such as at or in the bladder, bladder neck, urethra, kidney, ureter, etc. Sleeves 226 are illustrated at the external portion of shaft 222 of the device, sleeves 226 being capable of being moved independently from each other in a direction along the length of shaft 222, and covering one or more of delivery orifices 224. Each sleeve 226 can be mechanically actuated to move along a length of shaft 222 in a manner to allow one or more of delivery orifices 224 to be covered (closed) or uncovered (opened), and to allow fluid to be delivered from any of open delivery orifices 224. Drainage lumen 228 and drainage aperture 230 are illustrated. Drainage lumen 228, connected to drainage aperture 230, is illustrated to be located along a side portion of distal end 220, extending to a proximal end to allow drainage of urine from the bladder during installation of the device in the patient. Multiple fluid delivery means 232, including pressure source, reservoir, or both, can be located somewhere within distal end 220, e.g., next to each orifice 224. Each fluid delivery means 232 can include a pressure source located at the distal end of the device, or a pressure source may be located at the proximal end (not shown). A fluid delivery lumen (not shown) connects each fluid delivery means 232 to a proximal end of the device. Pressurization of each delivery means 232 can be individually controlled by an actuating mechanism located at the proximal end of the device that communicates either mechanically or electronically with a pressure source (at the proximal or distal end of the device) to pressurize a fluid for delivery at the distal end. The dashed line to each fluid delivery means 232 indicates an electronic or mechanical connection to a proximal end that allows for activation of fluid delivery means 232 to deliver fluid.

As used herein, the phrase “fluid delivery means” refers to one or more structures and mechanisms, alone or separately, that can be used to eject a fluid from a fluid delivery orifice, and includes, separately or in combination, items such as a pressure source, fluid reservoir, a lumen, and an actuating mechanism.

Optionally, distal end 220 can include features such as a steerable shaft, to allow end 220 to be steered (e.g., articulated) during installation and delivery of a fluid. Also optionally, distal end 220 can include an optical feature leading to a proximal end of the device to allow viewing of a delivery location during use and accurate placement of distal end 220, orifices 224, and fluid delivered through orifices 224.

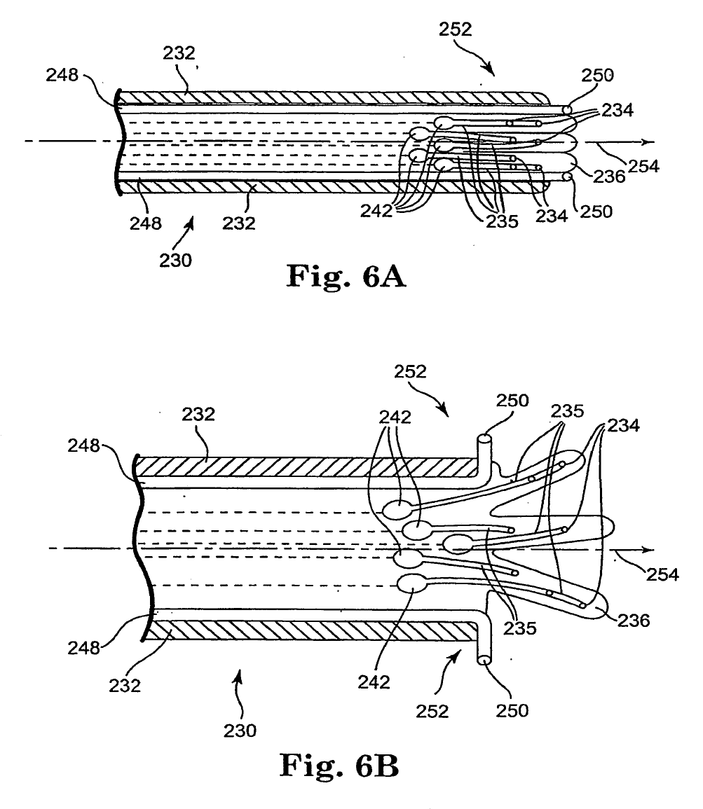

FIGS. 6A and 6B illustrate an embodiment of a device as described herein, which includes multiple, extendable fluid delivery orifices, i.e., fluid delivery orifices located at ends of orifice extensions. Referring to FIG. 6A, distal end 230 includes shaft 232, delivery orifices 234 at lengths or ends of orifice extensions 236. Delivery orifice extensions 236 include a flexible outer portion, and an extendable lumen 235 connected to a fluid delivery means 242, which can include a fluid reservoir, pressure source, or both. Each fluid delivery means 242 is connected, independently, to a proximal end by a mechanical or electronic connection to allow means 242 to be independently moved and actuated (connections shown by dashed lines). A fluid delivery lumen (not shown) connects each fluid delivery means 242 to a proximal end of the device. Each fluid delivery means 242 can control a single source and type of fluid so that multiple different types of fluid can be delivered from distal end 230, or, optionally, an identical fluid can be delivered using each different means 242 connected to different fluid sources that contain the identical fluid.

Extendable drainage lumens 248 connect to drainage apertures 250 and to a proximal end (not shown) of the device located at a proximal end of shaft 232. The drainage lumens can extend past tip 252 in a longitudinal direction, lateral direction, or both, and can act as a locating mechanism as well as a drainage lumen.

The multiple, extendable fluid delivery orifice extensions 236 are illustrated in a retracted position at FIG. 6A, wherein each delivery orifice extension 236 is retracted to be positioned within shaft 232 of distal end 230. Fluid delivery orifices 234 are located at ends of each extendable lumen 235 and as illustrated and at sides of some of extendable lumens 235. Each extension 236 includes an outer portion that can be flexible, rigid, or semi-rigid extension or “finger” that extends distally away from the proximal end of shaft 232 and the overall fluid delivery device, and distally and optionally laterally away from device distal end or tip 252. The extension 236 can be prepared of an elastomeric, polymeric material such as a silicone, a polyurethane, a rubber or latex, etc., or may be a more rigid material. As extensions 236 may be biased so that as extensions 236 are actuated to extend beyond and exit distal end tip 252 of the device, extensions 236 may splay or fan apart laterally when extended in a manner that provides distance between the distal ends of each extension 236; extensions 236 move distally and laterally, distally with a component that is in a direction away from tip 252 and along an imaginary longitudinal axis (254) of shaft 232, and laterally away from axis 254. The fanning or splaying apart of orifice extensions 236 also provides a predicable distance between delivery orifices 234 located at each extension 236.

A device of FIG. 6A may be useful, e.g., to locate multiple fluid delivery orifices within a urethra, within a bladder neck, or within a bladder, etc., to deliver a fluid or fluids, of the same or different types, to multiple locations at one or more of these tissues. FIG. 6B illustrates distal end 230 of such a device with extended fingers (i.e., extended orifice extensions) 236, each finger 236 including an extendable lumen 235 that extends along a portion of the total length of each finger 236 to allow fluid to be ejected from one or more fluid delivery orifice 234 at the end of or at a length along a finger 236. Drainage lumens 248 are also extended. Individual fluid delivery means 242 relative to each finger 236 and extendable lumen 235 can include a fluid reservoir containing fluid, and optional pressure source, and can be independently activated or pressurized from a proximal end by a mechanical or an electrical connection (shown as dashed lines). One or more fluid reservoir of each means 242 can be pressurized to cause fluid to be ejected from one or more orifices of each extended finger 236 upon pressurization. Pressure can be provided as desired, such as by use of compressed carbon dioxide located at the distal or proximal end of the device, or by a mechanical spring, solenoid, or pump, or another type of pressure source, at a proximal end of a device. Each fluid delivery means 242 can be controlled independently or together from a proximal end of the device (not shown), to independently deliver a single type of fluid.

Optionally, distal end 230 can include steerable distal end or tip to allow distal end 230 to be steered during installation and delivery of a fluid. Also optionally, distal end 230 can include an optical feature leading to and visually connecting to a proximal end of the device to allow viewing of a delivery location during use and accurate placement of distal end 230, orifices 234, and fluid delivered through orifices 234.

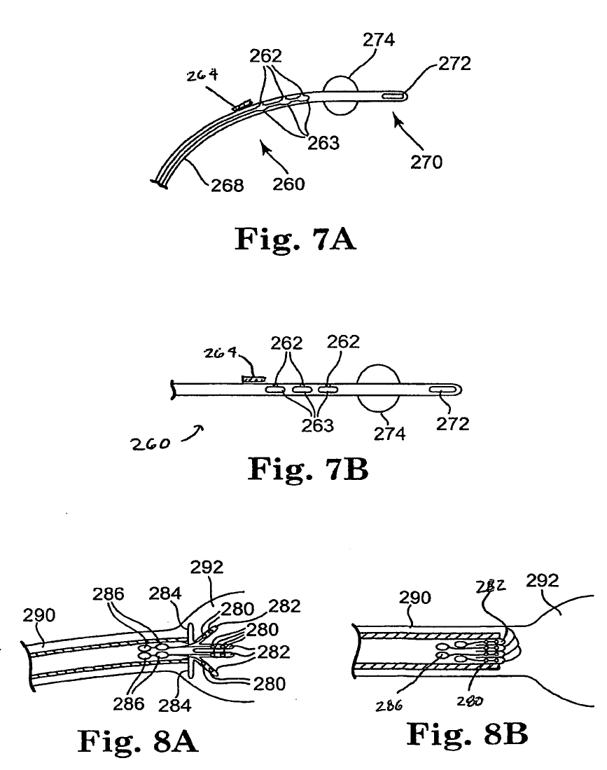

FIGS. 7A and 7B illustrate another embodiment of a distal end of a device according to the invention. FIG. 7A illustrates delivery orifices connected in series to deliver a single type of fluid from multiple lumens, i.e., a “daisy chain” configuration of multiple adjacent fluid delivery orifices along a length of a distal end of a device, located on a proximal side of a balloon. FIG. 7B illustrates a similar embodiment that instead includes “parallel” fluid delivery orifices that can each be independently controlled to deliver a different type of fluid (actuating mechanisms and fluid delivery lumens are not all shown).

FIG. 7A shows distal end 260 of a fluid delivery device, which includes fluid delivery orifices 262, cover 264 that can cover one or more of orifices 262 (e.g., needleless fluid delivery orifices) as desired, shaft 268, inflatable balloon 274, and drainage aperture 272 at tip 270 of distal end 260. Fluid delivery orifices 262 are located at progressively distal locations along the length of shaft 268, and may also be at adjacent positions around the perimeter of the shaft (as illustrated) or at different positions around the perimeter of the shaft. Balloon 274 may be for placement in a bladder to assist in properly locating distal end 260 during use. Drainage aperture 272 is at tip 270 of the device connects to a drainage lumen (not shown) extending through shaft 268 to a proximal end (not shown) of the device. One or more independently-controllable sleeves 264 can be used to expose each fluid delivery orifice 262 separately or in any desired combination to allow delivery of fluid for injection or instillation at desired combinations of locations along the length or around the perimeter of the installed device.

FIG. 7B is illustrative of a similar device, except that delivery orifices 262 are not connected to a common fluid source, in series, but each orifice 262 is connected to a different fluid delivery means 263, to allow for delivery of a different fluid. Three fluid delivery lumens (not shown) connect each of the three fluid delivery means 263 to a proximal end of the device.

Optionally, distal end 260 can include features such as a steerable distal end, to allow end 260 to be steered during installation and delivery of a fluid. Also optionally, distal end 260 can include an optical feature leading to a proximal end of the device to allow viewing of a delivery location during use and accurate placement of distal end 260, orifices 262, and fluid delivered through orifices 262.

FIGS. 8A and 8B illustrate another embodiment of a device that includes multiple delivery orifices located at mechanical orifice extensions (“fingers”) that extend and expand past the distal end of a shaft of a delivery device as described herein. Each extendable finger 282 includes one or multiple delivery orifices 280 in communication with a separate fluid means 286, each of which can be independently pressurized to eject a different fluid from its respective orifices for injection or instillation of multiple different fluids at tissue of the bladder, urethra, bladder neck, etc. Extendable locating mechanism 284 is an extendable structure that can be extended and retracted by a mechanism at a proximal end of the device, and may or may not include a drainage lumen. FIG. 8A shows fingers 282 extended and splayed when a device is installed in urethra 290 and bladder 292, and extended locating mechanisms 284. FIG. 8B shows fingers retracted for installation through urethra 290.

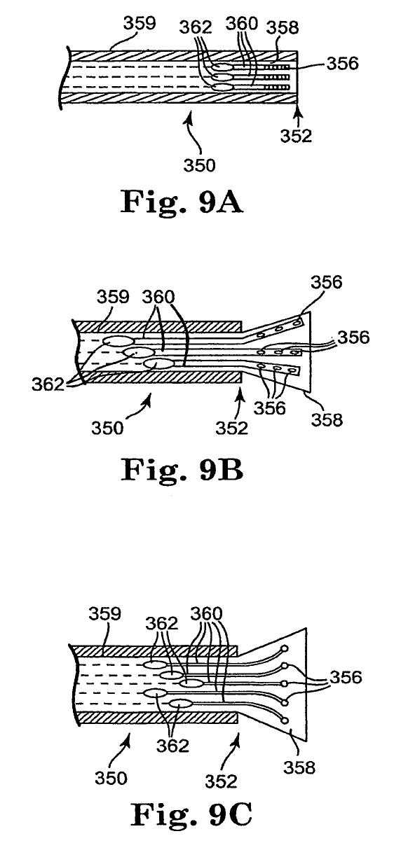

FIGS. 9A, 9B, and 9C illustrate another embodiment of a device that includes multiple fluid delivery orifices that are extendable or expandable in a direction that includes a component of distal direction, from a tip of a distal end of a device. This example includes orifice extension 358 in the form of an expandable “nozzle,” “funnel,” or “fan” configuration, such as a membrane, the expandable portion of the device containing multiple fluid delivery orifices 356, each orifice connected through a lumen 360 to a fluid reservoir or fluid delivery means 362 that may be pressurized to eject fluid from orifices of a single lumen 360. The extension 358 can be prepared of an elastomeric, polymeric material such as a silicone, a polyurethane, a rubber or latex, etc., or may be a more rigid material. Extendable lumens 360 are contained by extension 358, and each includes multiple orifices 356. As illustrated in FIGS. 9A and 9B, different orifices 356 are connected to separate fluid sources and so the device is able to deliver multiple different fluids or types of fluids. Alternately, all orifices 356 could be connected to a single fluid source for delivery of a single source of fluid.

Referring to FIG. 9A, distal end 350 of a fluid delivery device includes an open or hollow tip 352 of shaft 359, which further contains moveable orifice extension 358. Orifice extension 358 is shown at FIG. 9A in a retracted condition, contained by shaft 359. Orifice extension 358 is shown at FIG. 9B in an expanded condition, extending distally and laterally from tip 352. As shown in FIG. 9B orifice extension 358 is in the form of a membrane that fans out in three dimensions when extended from tip 352. The membrane includes fluid delivery orifices 356 that may be connected to a fluid reservoir, either a distal reservoir (362, as illustrated, located at a distal end of the fluid delivery device) or a proximal reservoir (located at a proximal end of the fluid delivery device). Fluid delivery lumens (not shown) connect each reservoir or means 362 to a proximal end. Dashed lines indicate a mechanical or electronic mechanism to separately and independently activate each reservoir or means 362; each reservoir 362 communicates with a proximal end of a device to allow independent movement or actuation of a reservoir or means 362, and independent fluid delivery. A fluid delivery lumen 360 extends from each means or reservoir 362 to each fluid deliver orifice 356. Orifice extension 358 also is connected to the proximal end to allow actuation or movement of extension 358 from a location at the proximal end.

FIG. 9C illustrates an alternate embodiment that includes one fluid delivery means 362 per single fluid delivery orifice 356.

Optionally, for a device as shown at FIGS. 9A, 9B, and 9C, a fluid reservoir may be at the distal end of the device or the proximal end, and a pressurization mechanism (e.g., carbon dioxide, a plunger, etc.) may also be at a proximal end or a distal end of the device. Orifice extension (fan) 358 containing fluid delivery orifices 356 extends and expand past distal end tip 352 of shaft 354 of the delivery device, and can be actuated to expand by an orifice extension actuating mechanism at a proximal end of the device. Orifice extension (fan) 358 can include one or multiple delivery orifices 356 at various positions along the length or circumference of fan 358, as fan 358 is in an extended or expanded position. FIGS. 9B and 9C illustrate expanded states of fan 358, showing the fan or “funnel” shape and exemplary configurations of fluid delivery orifices 356, lumens 360 between each orifice 356 and fluid delivery means or reservoir (e.g., 362), and pressure mechanisms (not shown). Each orifice 356 off an 358 is in communication with a fluid reservoir (e.g., 362, either at the proximal or distal end of the device) that can be pressurized to eject fluid from each orifice 356 for injection or instillation of tissue of the bladder, urethra, kidney, ureter, bladder neck, etc.

Optionally, distal end 350 can include features such as a steerable distal end, to allow end 350 to be steered during installation and delivery of a fluid. Also optionally, distal end 350 can include an optical feature leading to a proximal end of the device to allow viewing of a delivery location during use and accurate placement of distal end 350, orifices 356, and fluid delivered through orifices 356.

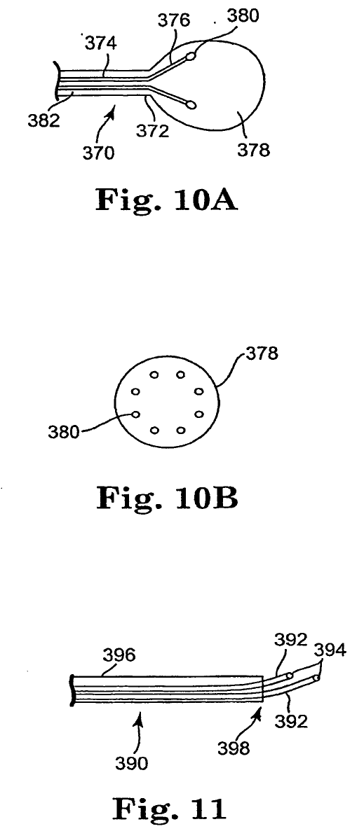

FIGS. 10A and 10B illustrate yet another embodiment of a device that includes multiple delivery orifices that are extendable or expandable from a distal end of the device. Referring to FIG. 10A, distal end 370 of a fluid delivery device includes an open or hollow tip 372 of shaft 382, and orifice extension 378. Orifice extension 378, which is a balloon at tip 372 of shaft 382, is shown at FIG. 10A in an expanded condition, extending distally and laterally from tip 372. Extension 378 can be prepared of an elastomeric, polymeric material such as a silicone, a polyurethane, a rubber or latex, etc., or may be a more rigid material. FIG. 10A is a side view of a device with two delivery orifices 380. Each orifice 380 is connected to a different fluid delivery lumen 374, and may deliver a different fluid. Alternately each orifice could be connected to the same fluid source, or additional orifices may be present

FIG. 10B is an end view of a configuration similar to that of FIG. 10A, but including 8 orifices 380. Each orifice 380 may be connected to a different fluid delivery lumen 374 to deliver a different fluid, or two or more orifices could be connected to the same fluid source. Extension portion 378 is in the form of a balloon that expands in three dimensions when inflated. Balloon 378 includes fluid delivery orifices 380 that may be connected to a fluid reservoir, either a distal reservoir at a distal end of the fluid delivery device or a proximal reservoir located at a proximal end of the fluid delivery device. A fluid delivery lumen may extend from the reservoir to each fluid deliver orifice 380.

The exemplary device of FIGS. 10A and 10B includes an expandable balloon configuration, the expandable balloon containing multiple fluid delivery orifices, each orifice connected to a fluid reservoir (not shown) that may be pressurized to eject fluid from a single orifice. The fluid reservoir may be at the distal end of the device or the proximal end, and a pressurization mechanism (e.g., carbon dioxide, a plunger, etc.) may also be at a proximal end or a distal end of the device. Balloon 378, containing fluid delivery orifices 380 expands upon inflation using an inflation fluid that may be delivered from a proximal end of the device through an inflation lumen connecting the balloon to the proximal end. The balloon expands, which causes the fluid delivery orifices to move and become located beyond the distal end of the shaft of the delivery device. The balloon can be expanded inside of the bladder in a manner to fill or partially fill and to cause the fluid delivery orifices to contact internal tissue of the bladder. As the balloon expands, fluid delivery orifices extend away from one another along with the balloon surface and become located near the surface of the bladder internal tissue. Each orifice of the balloon can be located at any desired distance or radial location from the end of the shaft, such as uniformly around the balloon as shown in FIG. 10B or in another pattern of various distance from the base of the balloon at the device shaft, or randomly. Orifices can be at the same or different distances from the end of the shaft.

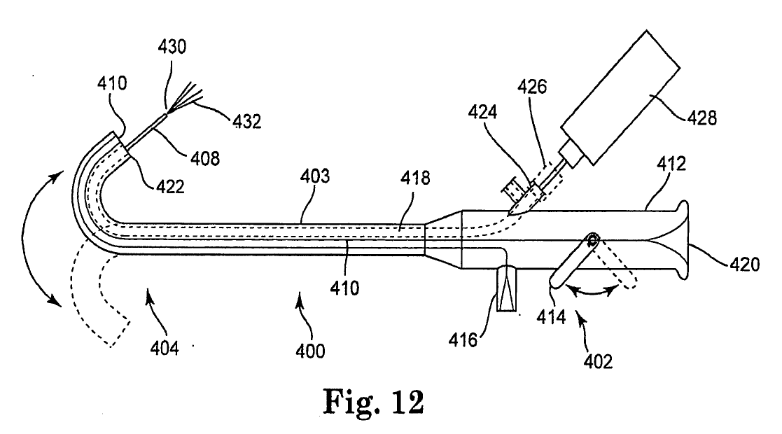

FIG. 11 illustrates yet another embodiment of a device that includes one or multiple (as illustrated) delivery orifices that are extendable from a distal end of the device. Referring to FIG. 11, distal end 390 of a fluid delivery device includes distal tip 398 of shaft 396, which further contains orifice extensions 392. Orifice extensions 392 are in the form of extendable lumens or “fingers” that extend distally from tip 398 of shaft 396. Extendable orifice extensions 392 or “fingers” include fluid delivery orifices 394 at their distal ends. Each orifice extension 392 or “finger” may be steerable using an actuating mechanism at a proximal end of the device, e.g., by use of a steering mechanism discussed above for a steerable shaft. Also, each orifice extension 392 may be biased, if desired, for biased extension distally from tip 398, by manipulation of an actuating mechanism at a proximal end of the device. Each orifice 394 can be connected to a fluid reservoir that may be pressurized to eject fluid from a single orifice. The fluid reservoir may be at the distal end of the device or the proximal end, or remote from a proximal end, and a pressurization mechanism (e.g., carbon dioxide, a plunger, a mechanical spring, a pump, a solenoid, etc.) may also be at a proximal end or a distal end of the device.

According to particular embodiments of the invention, distal end 390 can include features such as a steerable distal end to allow end 390 to be steered during installation and delivery of a fluid. Also optionally, distal end 390 can include an optical feature leading to a proximal end of the device to allow viewing of a delivery location during use and accurate placement of distal end 390, orifices 394, and fluid delivered through orifices 394.

Another exemplary embodiment of a device according to the invention is illustrated at FIG. 12. Device 400 includes proximal end 402 and distal end 404, connected by shaft 403. Proximal end 402 includes body or handle 412, which contains features useful for manipulating or operating features at distal end 404. Body 412 includes: fiber optic light source 416; steering actuator 414, which can be manipulated to cause the steerable distal end of device 400 to move in two dimensions based on differential pushing and pulling (or tension and compression) of multiple cables within walls of the shaft (not shown); viewing lens 420 that allows viewing through fiber optic cable 410 to a location of fluid delivery past tip 422; and port 424, which allows for connection of a fluid source to proximal end 402. Articulation for steering of end 404 is indicated in dashed lines.

Still referring to FIG. 12, mounting adapter 426 attaches to port 424 to allow fluid delivery means 428 to connect to body 412. Fluid delivery means 428 includes a fluid reservoir and pressure source, and mechanisms for causing the pressure source to pressurize the fluid reservoir to cause fluid to flow from the fluid reservoir, through port 424, through fluid lumen 408 (which is longitudinally moveable within working lumen 418), and to ultimately exit fluid lumen 408 at fluid delivery orifice 430 as fluid jet injection 432. An actuator (not shown) at proximal end 402 can be manipulated to allow extendable fluid lumen 408 to be distally extended and retracted.

Body 412 connects to shaft 403, which includes lumens and mechanisms that connect features of proximal end 402 to distal end 404. Working lumen 418 is a hollow lumen or channel that extends within shaft 403 and contains fluid delivery lumen 408 in a manner that allows fluid delivery lumen 408 to move longitudinally along the length of shaft 403, to allow the distal end of fluid delivery lumen 408 to extend from tip 422 as an orifice extension. Shaft 403 also includes fiber optic 410 and a steering mechanisms (not shown) that allows steering (deflecting) of distal end 464 by movement of actuator 414. Light source 416 transmits light to distal end 404 by fiber optic 411.

Distal end 404 includes tip 422 from which can be extended fluid delivery lumen 408 as an orifice extension. Also distal end 404 can be steered in two dimensions to allow movement of tip 422, in coordination with extension of lumen 408, based on viewing through fiber optic 410, to deliver a fluid with accurate placement at a desired tissue location. Optionally, another steerable section of shaft 403 could be included, e.g., proximal to the illustrated steerable section, to allow distal end to articulate in an additional plane, such as a plane that is perpendicular to the plane of articulation illustrated at FIG. 12.

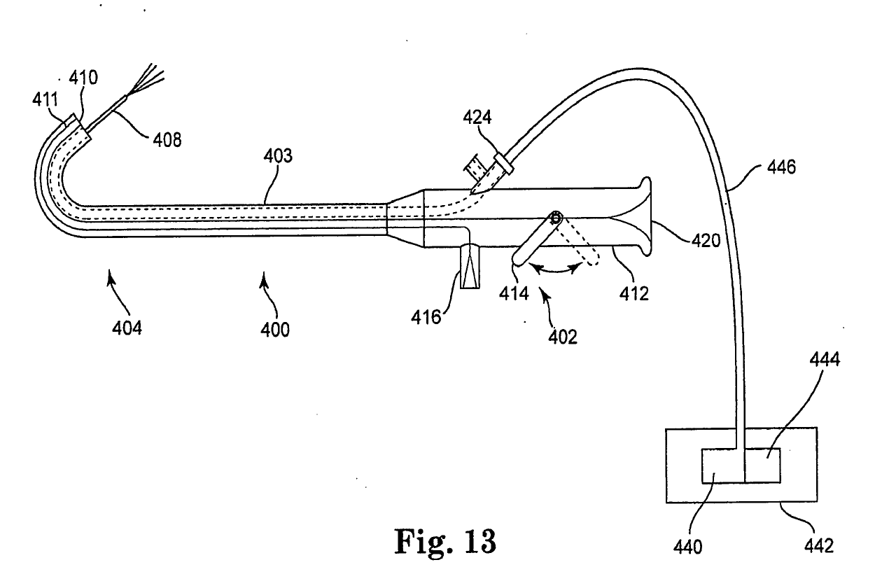

A modification of device 400 is illustrated at FIG. 13. In FIG. 13, port 424 is not connect to a proximal fluid delivery means 428 as shown in FIG. 12, but is connected to a remote fluid source 440. In specific, device 400 connects through port 424 to flexible conduit 446, which includes a fluid lumen to allow fluid communication between port 424 and fluid reservoir 440. Fluid reservoir 440 is contained within remote console 442, which contains fluid reservoir 440 and pressure source 444 capable of pressurizing fluid contained by fluid reservoir 440. “Remote” console 442 can be a desirable distance from body 412 to allow for easy manipulation and use of device 440 but with a relocation of weight and bulk of a fluid reservoir and pressure source away from body 412. A “remote” console may be located, for example, from 2 feet to 20 feet from the user of device 400, and flexible conduit 446 may be a commensurate length, such as from 4 feet to 30 feet in length.

The use of a console as shown in FIG. 13 is not limited to usefulness in combination with a device having the particular features of device 400. A remote console such as console 442 can be used with any of the devices or features described herein or shown in any of FIGS. 1 through 11.

FIGS. 14 and 15 illustrate a delivery volume control apparatus (500) that may be incorporated into any of the fluid delivery devices described herein. Delivery volume control apparatus 500 generally comprises a plunger shaft assembly 502 and a stop member 504. Delivery volume control 500 can be fabricated of appropriate material including metals such as stainless steel and nitinol or alternatively, suitable polymeric materials. Plunger shaft assembly 502 can comprise a shaft member 506 and a graduated engagement member 508. Graduated engagement member 508 is generally circumferentially disposed about the shaft member 506 and can include a first engagement portion 510, a second engagement portion 512 and a third engagement portion 514. Each of the engagement portions has a distinct diameter that decreases from the first engagement portion 510 to the second engagement portion 512 and finally to the third engagement portion 514. Each engagement portion includes an engagement surface such as a first engagement surface 510a on the first engagement portion 510, a second engagement surface 512a on the second engagement portion 512 and a third engagement surface 514a on the third engagement portion 514. Shaft member 506 generally extends from the third engagement portion 514 to a plunger (not shown) at the end of shaft assembly 502 that is opposite of graduated engagement member 508.

The plunger can pressurize and displace fluid in a plunger reservoir. As will be understood, the distance of movement of a plunger at the end of can be controlled by movement of stop member 504 to engage different surfaces of engagement member 508, which can control a volume of fluid displaced by the plunger.

Stop member 504 generally includes a stop body 516 defining a stop surface 518. Stop member 504 can be operably mounted within shaft 507. Stop member 504 is generally configured for retainable placement into surface opening 509 through the use of suitable retention mechanisms including, for example, a friction fit, magnetic coupling, detent means, ratcheted surfaces, spring-loaded retention members, and the like.

In use, the stop member 504 is biased into surface opening 509 of shaft 507 such that a desired amount of stop surface 518 is present within shaft 507. The amount of stop surface 518 within shaft 507 is selected based on which of the first engagement surface 510a, the second engagement surface 512a or the third engagement surface 514a is desired to be engaged. By selecting which engagement surface is to be engaged, the stroke length of the plunger shaft assembly 502 is limited such that each full stroke delivers the same measured amount of fluid upon movement of a plunger. Through selective placement of stop member 504, a medical professional can vary the volumetric amount of therapeutic fluid that is ultimately administered with each stroke of plunger shaft assembly 502.

Any individual component of a device, or an entire device, could be disposable or reusable. As an example, a disposable or reusable optical feature such as of the type used with endoscope devices could be incorporated into a portion or component of a device that is either disposable or reusable. Additionally or alternately, a device could have a reusable pressure source (e.g., cartridge of pressurized gas), a replaceable fluid reservoir, a disposable delivery head portion, or may be entirely disposable.

In another particular embodiment, a device could be designed to deliver multiple volumes of fluid at different tissue locations. Accordingly, the fluid deliveries may be made between steps of relocating a fluid delivery orifice, wherein one or more fluid delivery orifices may optionally be moveable relative to the shaft or extendable from the shaft. The multiple volumes of fluid could be pre-loaded into individual, e.g., replaceable, vials of a predetermined volume as desired for a single or multiple fluid deliveries, i.e., a single vial may include a single dose (volume) or multiple doses (volumes) of fluid. With the use of a replaceable vial, the device could be used to deliver one or multiple doses of fluid using the entire volume from one vial. The replaceable vial could then be removed from the fluid delivery device and replaced with a full vial, and the volume of the full vial could be delivered in one or more fluid deliveries. This could be repeated for as many fluid deliveries as desired. In alternate embodiments, the device may have a connection at the proximal end for connecting the device to a remote source of fluid (e.g., a “hopper” or remote console) from which multiple fluid deliveries of desired volumes could be sourced without the need for loading or reloading individual vials.

With any of the above features of fluid delivery devices, a device could include an electronic process control system that can be programmed to make fluid deliveries having various locations, volumes, and other injection properties such as depth and degree (e.g., shape and distance) of dispersion and size of particles of fluid.

The invention also provides methods of delivering fluid to tissue of the lower urinary tract, and nearby tissues, e.g., tissue of the bladder or bladder neck, kidney, ureter, urethra, prostate, including the steps of: providing a fluid source and a fluid delivery device substantially as described herein; inserting the fluid delivery device into the patient, e.g., through the meatus and into the urethra; navigating the device until a fluid delivery orifice at a distal end of the device is positioned at a desired delivery site; optionally actuating a feature of the distal end of the device such as opening a cover or port to expose a fluid delivery orifice, expanding or extending a fan, finger, balloon, or other extension of the device that includes a fluid delivery orifice, and actuating the device to deliver one or multiple types of fluid from one or multiple orifices at the site. Multiple delivery orifices can provide the ability to place one or more different fluids at multiple locations of the urethra, prostate, bladder, or bladder neck, etc. Features of devices described herein, such as optical features, steerable shafts, extendable or moveable fluid delivery orifices, the ability to independently open and close selected fluid delivery orifices, and the ability to deliver multiple different types of fluid, allow for improved control over the location of injection or instillation of a fluid.

Exemplary methods of treatment can include one or multiple discrete steps relating to insertion of a fluid delivery device as described herein; positioning of the device to place one or more fluid delivery orifices at desired locations within the bladder or bladder neck or other location of the urinary tract; optionally, use of an optic device; optional extension of a needle or a needleless delivery orifice extension from the shaft of the device to contact tissue of the bladder or bladder neck, etc.; delivery of one or more biologically active fluid or agent from a delivery orifice (needle or needleless delivery orifice) to either contact or penetrate tissue of the bladder or bladder neck, etc.; optionally, one or multiple steps of re-positioning one or more fluid delivery orifices; optionally, one or more additional delivery steps that involve the same or different delivery orifices.

According to fluid delivery procedures of the invention, fluid such as ethanol or a biologically active agent can be delivered to the bladder, urethra, or bladder neck, etc., in a manner that causes the fluid to be injected into the tissue using a needle or a needleless delivery orifice, or that causes the fluid to contact a surface of the tissue, allowing a biologically active component of the fluid to absorb through the tissue.

Devices of the present description can be useful to treat of tissue of the urinary tract in females or males. For example, devices of the invention may be useful to inject the bladder, bladder neck, the urethral tissue itself or the external sphincter, or for transurethral injection of the prostate in a male. In other embodiments, a fluid may be injected into tissue of the urinary tract (e.g., bladder, urethra, kidneys, ureters, prostate, etc.) such as individual or combination treatments using drugs or other therapeutic agents, e.g., botulism toxin (“botox”), an antiandrogen, among others as will be understood. One advantage of injection of an active pharmaceutical agent at a location of use is the placement of the agent to avoid systemic side effects. Specific examples of active pharmaceutical agents that may be injected include Botulism Toxin types A through G; 5-alpha reductase inhibitors such as dutasteride and finasteride; alpha blockers such as alfuzosin, doxazosin, prazosin, tamsulosin hydrochloride, terazosin, to treat BPH; or any of various antibiotics (e.g., to treat prostatitis) and analgesics.

Methods of using a device can include delivery of one or more therapeutic agent, e.g., multiple agents such as multiple drugs, to tissue of the urinary tract, e.g., bladder, bladder neck, urethra, prostate, kidney, ureter, etc. According to certain exemplary methods the fluid is injected into tissue. According to other exemplary methods the fluid is instilled to contact a surface of a tissue, e.g., to wash the tissue (such as to fill the bladder to wash bladder tissue) or to deliver a therapeutic agent. According to still other exemplary methods, fluid can be delivered into the bladder to fill or partially fill the bladder interior.

Other embodiments of this invention will be apparent to those of ordinary skill upon consideration of this description or from practice of the invention described and illustrated herein. Various omissions, modifications, and changes to the principles and embodiments described herein may be made by one skilled in the art without departing from the true scope and spirit of the invention which is indicated by the following exemplary embodiments of devices.

1. A device for delivery of fluid to tissue of the urinary tract, the device comprising:
a proximal end,
a flexible shaft extending from the proximal end to a distal end of the shaft,
an extendable fluid delivery orifice at the distal end, the fluid delivery orifice being capable of being extended from the shaft, and
a pressure source capable of pressurizing fluid and ejecting fluid from the fluid delivery orifice with a pressure capable of penetrating tissue.

2. The device of claim 1 comprising a body at the proximal end, the body comprising the pressure source and a fluid reservoir, the fluid reservoir being in fluid communication with the fluid delivery orifice, the pressure source being capable of pressurizing the fluid reservoir.

3. The device of claim 1 comprising a remote console in communication with the proximal end, the remote console comprising the pressure source and a fluid reservoir, the fluid reservoir being in fluid communication with the fluid delivery orifice, the pressure source being capable of pressurizing the fluid reservoir.

4. The device of claim 1 comprising a paddle extension at a distal end of the shaft, the paddle extension comprising an attached end connected to the shaft and a moveable end that can be extended laterally from the shaft, wherein a fluid delivery orifice is located at the moveable end.

5. The device of claim 1 comprising a fluid delivery orifice extension capable of being extended from a distal end of the shaft in a direction that includes one direction component along a longitudinal axis of the shaft at the distal end, and a second direction component lateral from the longitudinal axis.

6. The device of claim 5 wherein the fluid delivery orifice extension is an expandable balloon at a distal end of the shaft, and the device comprises multiple fluid delivery orifices attached to the expandable balloon.

7. The device of claim 1 comprising a drainage lumen extending from the distal end to the proximal end, capable of draining urine from a bladder when the device is installed in a patient.

8. The device of claim 7 comprising a steerable shaft distal end.

9. The device of claim 1 comprising optics to allow optical communication between the proximal end and a view from the distal end.

10. The device of claim 1 wherein the distal end of the shaft is steerable.

11. The device of claim 10 comprising optics to allow optical communication between the proximal end and a view from the distal end.

12. The device of claim 10 wherein the fluid delivery orifice is an extendable fluid delivery lumen that extends distally from the tip of the shaft.

13. The device of claim 12 comprising a remote console connected through a flexible conduit to the proximal end, the console comprising the pressure source and a fluid reservoir in communication with a fluid delivery orifice, the pressure source being capable of pressurizing the fluid reservoir.

14. A device of claim 1, capable of independently delivering fluid from multiple orifices, the device comprising
two fluid delivery orifices at the distal end, and
two fluid reservoirs,
wherein one fluid reservoir is in fluid communication with one fluid delivery orifice and the other fluid delivery reservoir is in fluid communication with the other fluid delivery orifice.

15. A method of delivering fluid to tissue of the urinary tract, the method comprising,
providing a device comprising
a proximal end,
a flexible shaft extending from the proximal end to a distal end of the shaft,
an extendable fluid delivery orifice at the distal end, the fluid delivery orifice being capable of being extended from the shaft,
inserting the distal end into the urethra to place a fluid delivery orifice at a location of the urinary tract,
extending an extendable fluid delivery orifice, and
delivering fluid to the urinary tract.

16. The method of claim 15 comprising injecting the fluid into tissue of the lower urinary tract.

17. The method of claim 15 comprising instilling the fluid to a surface of tissue of the lower urinary tract.

18. The method of claim 15 wherein the tissue is selected from the group consisting of: urethral tissue, prostate tissue, bladder neck tissue, bladder tissue, ureter tissue, and kidney tissue.

19. A device for delivery of fluid to tissue of the urinary tract, the device comprising:
a proximal end,
a shaft extending from the proximal end to a distal end, the shaft comprising a steerable portion,
a fluid delivery orifice at the distal end, and
optics to allow optical communication between the proximal end and the distal end.

20. The device of claim 19 comprising multiple fluid delivery systems, each system comprising a fluid reservoir and a fluid delivery orifice.

21-24. (canceled)

25. The device of claim 1 wherein the pressure is 2000 pounds per square inch or greater.

26-27. (canceled)

28. The device of claim 19 comprising a pressure source capable of pressurizing fluid and ejecting fluid from the fluid delivery orifice with a pressure capable of penetrating tissue.

29. The device of claim 19 wherein the pressure is 2000 pounds per square inch or greater.
